(19) United States
(12) Patent Application Publication
(10) Pub. No.: US 2026/0048174 A1
(43) Pub. Date: Feb. 19, 2026

(54) DEVICES AND METHODS FOR AMELIORATING IMPLANT-INDUCED INFLAMMATION

(71) Applicants: Cerapedics, Inc., Westminter, CO (US); Allegheny-Singer Research Institute, Pittsburgh, PA (US)

(72) Inventors: Jerome CONNOR, Doylestown, PA (US); Boyle C. CHENG, Mars, PA (US)

(21) Appl. No.: 19/102,903

(22) PCT Filed: Aug. 11, 2023
(86) PCT No.: PCT/US2023/072095

§ 371 Date: Feb. 11, 2025

Related U.S. Application Data
(60) Provisional application No. 63/397,236, filed on Aug. 11, 2022.

(51) Int. Cl. A61L 27/18; A61F 2/30; A61F 2/44; A61F 2/82; A61L 27/22; A61L 27/24; A61L 27/34; A61L 27/46; A61L 27/52; A61L 27/56

(52) U.S. Cl. CPC A61L 27/18; A61F 2/4455; A61F 2/82; A61L 27/227; A61L 27/24; A61L 27/34; A61L 27/46; A61L 27/52; A61L 27/56; A61F 2002/3006; A61F 2210/0014; A61L 2300/252; A61L 2300/41; A61L 2400/18; A61L 2430/02; A61L 2430/34; A61L 2430/38

(57) ABSTRACT

This invention features methods and devices for ameliorating implant-induced inflammation and for reducing the risk of fibrosis. The methods and devices include a substrate coated with P-15 peptide positioned at the site of implantation.

### SEQUENCE LISTING

The instant application contains a Sequence Listing which has been submitted electronically in XML format and is hereby incorporated by references in its entirety. The XML copy, created on Aug. 10, 2023, is named “50517-031WO2_Sequence_Listing_8_10_23” and is 1,783 bytes in size.

### BACKGROUND OF THE INVENTION

Surgical implantation of medical devices, regardless of the implant being made of inert and nontoxic materials, will initiate the on-set of a foreign body reaction (FBR) (see Bridges et al., J Diabetes Sci Technol. 2(6):984 (2008)). It has been established that the combination of at least two events contribute to triggering an inflammatory cascade by the host—the immediate adsorption and binding of blood plasma proteins and other molecules onto the implant's surface, as well as the injury of the micro-vasculature and tissue during implantation and the associated histamine release by local mast cells. The progression of FBR, including leukocyte recruitment, differentiation into foreign body giant cells (FBGCs), and cytokine and matrix metalloproteinases (MMPs) release, can be features of an acute host inflammatory response to the implantation of a device, leading to implant-induced fibrosis and, ultimately, implant failure.

Methods to mediate inflammatory events associated with implant-induced inflammation are needed.

### SUMMARY OF THE INVENTION

The invention features a method for fixing two vertebral bodies of a subject, the method including: (a) providing (i) a spinal fusion cage including a polyarylether ketone (PAEK) having an internal surface and an external surface, and (ii) inorganic particles coated with P-15 peptide; and (b) positioning (i) the spinal fusion cage, and (ii) the inorganic particles coated with P-15 peptide between a first vertebral body and a second vertebral body; wherein step (b) including placing the inorganic particles coated with P-15 peptide within the spinal fusion cage and outside of the spinal fusion cage.

In particular embodiments, the PAEK is a polyether-ether-ketone (PEEK), a polyetherketone (PEK), a polyetherketoneketone (PEKK), a polyetheretherketoneketone (PEEKK), or a poly(aryl-ether-ketone-ether-ketoneketone (PEKEKK).

In some embodiments, the PAEK has a molecular weight (Mn) of from 110-120 KDa, a molecular weight (Mn) of from 100-110 KDa, or a molecular weight (Mn) of from 80-100 KDa.

In certain embodiments, the PAEK has a glass transition temperature of between 250° C. and 450° C. For example, the PAEK can have a glass transition temperature of between 300° C. and 380° C., 340±20° C., 365±5° C., or 375±5° C.

In particular embodiments, the PAEK is a composite material including fibers (e.g., carbon fibers) and/or a radio-opacity agent (e.g., barium sulfate).

The invention further features a method for fixing two vertebral bodies of a subject, the method including: (a) providing (i) a spinal fusion cage including a polyetherketoneketone (PEKK) having an internal surface and an external surface, and (ii) inorganic particles coated with P-15 peptide; and (b) positioning (i) the spinal fusion cage, and (ii) the inorganic particles coated with P-15 peptide between a first vertebral body and a second vertebral body. In particular embodiments, the PEKK has a glass transition temperature of between 250° C. and 450° C. (e.g., between 300° C. and 380° C., 340±20° C., 365±5° C., or 375±5° C.). In some embodiments the PEKK has a molecular weight (Mn) of from 110-120 KDa; from 100-110 KDa, or from 80-100 KDa. In particular embodiments, the PEKK is a composite material including fibers (e.g., carbon fibers) and/or a radio-opacity agent (e.g., barium sulfate).

The invention also features a method for fixing two vertebral bodies of a subject, the method including: (a) providing (i) a spinal fusion cage including a polyether-ether-ketone (PEEK) having an internal surface and an external surface, and (ii) inorganic particles coated with P-15 peptide; and (b) positioning (i) the spinal fusion cage, and (ii) the inorganic particles coated with P-15 peptide between a first vertebral body and a second vertebral body. In particular embodiments, the PEEK has a glass transition temperature of between 250° C. and 450° C. (e.g., between 300° C. and 380° C., 340±20° C., 365±5° C., or 375±5° C.). In some embodiments the PEEK has a molecular weight (Mn) of from 110-120 KDa; from 100-110 KDa, or from 80-100 KDa. In particular embodiments, the PEEK is a composite material including fibers (e.g., carbon fibers) and/or a radio-opacity agent (e.g., barium sulfate).

The invention further features a method for fixing two vertebral bodies of a subject, the method including: (a) providing (i) a spinal fusion cage including a polyetherketone (PEK) having an internal surface and an external surface, and (ii) inorganic particles coated with P-15 peptide; and (b) positioning (i) the spinal fusion cage, and (ii) the inorganic particles coated with P-15 peptide between a first vertebral body and a second vertebral body. In particular embodiments, the PEK has a glass transition temperature of between 250° C. and 450° C. (e.g., between 300° C. and 380° C., 340±20° C., 365±5° C., or 375±5° C.). In some embodiments the PEK has a molecular weight (Mn) of from 110-120 KDa; from 100-110 KDa, or from 80-100 KDa. In particular embodiments, the PEK is a composite material including fibers (e.g., carbon fibers) and/or a radio-opacity agent (e.g., barium sulfate).

The invention also features a method for fixing two vertebral bodies of a subject, the method including: (a) providing (i) a spinal fusion cage including a polyetheretherketoneketone (PEEKK) having an internal surface and an external surface, and (ii) inorganic particles coated with P-15 peptide; and (b) positioning (i) the spinal fusion cage, and (ii) the inorganic particles coated with P-15 peptide between a first vertebral body and a second vertebral body. In particular embodiments, the PEEKK has a glass transition temperature of between 250° C. and 450° C. (e.g., between 300° C. and 380° C., 340±20° C., 365±5° C., or 375±5° C.). In some embodiments the PEEKK has a molecular weight (Mn) of from 110-120 KDa; from 100-110 KDa, or from 80-100 KDa. In particular embodiments, the PEEKK is a composite material including fibers (e.g., carbon fibers) and/or a radio-opacity agent (e.g., barium sulfate).

The invention further features a method for fixing two vertebral bodies of a subject, the method including: (a) providing (i) a spinal fusion cage including a polyaryl-ether-ketone-ether-ketoneketone (PEKEKK) having an internal surface and an external surface, and (ii) inorganic particles coated with P-15 peptide; and (b) positioning (i) the spinal fusion cage, and (ii) the inorganic particles coated with P-15 peptide between a first vertebral body and a second vertebral body. In particular embodiments, the PEKEKK has a glass transition temperature of between 250° C. and 450° C. (e.g., between 300° C. and 380° C., 340±20° C., 365±5° C., or 375±5° C.). In some embodiments the PEKEKK has a molecular weight (Mn) of from 110-120 KDa; from 100-110 KDa, or from 80-100 KDa. In particular embodiments, the PEKEKK is a composite material including fibers (e.g., carbon fibers) and/or a radio-opacity agent (e.g., barium sulfate).

In the above methods, the inorganic particles can be selected from hydroxyapatite particles, dahllite particles, tetracalcium phosphate particles, calcium pyrophosphate particles, tricalcium phosphate particles, calcium hydrogen phosphate particles, octacalcium phosphate particles, calcium fluorapatite particles, and mixtures thereof. For example, the inorganic particles can be hydroxyapatite particles having diameters between 250 microns to 425 microns, such as anorganic bone mineral coated with P-15 peptide.

In embodiments of any of the above methods, the spinal fusion cage includes a porous material; the amount of the P-15 peptide bound to the surface of the inorganic particles is from 100 to 1500 ng of P-15 peptide per gram of inorganic particles; and/or the inorganic particles coated with P-15 peptide are suspended in a collagen hydrogel. In some embodiments, the weight ratio of the inorganic particles coated with P-15 peptide to the collagen is from 50:50 to 95:5. In a particular embodiment, the amount of the P-15 peptide bound to the surface of the inorganic particles is from 200 to 1200 ng of P-15 peptide per gram of inorganic particles, and the weight ratio of the inorganic particles coated with P-15 peptide to the collagen is from 75:25 to 95:5.

In one embodiment of any of the methods of the invention, placement of P-15 peptide in or around the spinal fusion cage reduces local inflammation between two vertebral bodies.

In another embodiment of any of the methods of the invention, placement of P-15 peptide in or around the spinal fusion cage reduces local fibrosis between two vertebral bodies.

The invention features a method of ameliorating implant-induced inflammation at an implantation site in a subject, the method including administering to the implantation site (i) an implantable medical device; and (ii) a substrate coated with P-15 peptide, wherein the substrate is not a calcified substrate.

The invention further features a method of ameliorating implant-induced inflammation at an implantation site in a subject, the method including inserting at the implantation site (i) an implantable medical device; and (ii) a substrate coated with P-15 peptide, wherein the implantation site does not include bone tissue.

The invention also features a method of ameliorating implant-induced inflammation at an implantation site in a subject, the method including inserting at the implantation site (i) an implantable medical device including a biodegradable polymer; and (ii) a substrate coated with P-15 peptide. In particular embodiments, the biodegradable polymer is selected from poly(lactic-co-glycolic acid) (PLGA), poly(lactic acid) (PLA), polycaprolactone (PCL), polylactic acid (PLA), polyglycolic acid (PGA), poly(butylene succinate) (PBS), and sucrose acetate isobutyrate (SAIB).

The invention features a method of ameliorating implant-induced inflammation at an implantation site in a subject, the method including inserting at the implantation site (i) an implantable medical device; and (ii) a substrate coated with P-15 peptide, wherein the implantation site is a soft tissue (e.g., muscle tissue, fat issue, connective tissue, organ tissue, subcutaneous tissue, ocular tissue, or brain tissue). In particular embodiments, the implantable medical device is implanted into a soft tissue selected from neurological tissue, vascular tissue, oral tissue, ocular tissue, nasal tissue, urogenital tissue, gastrointestinal tissue, biliary tissue, aural tissue, or subcutaneous tissue.

The invention further features a method of ameliorating implant-induced inflammation at an implantation site in a subject, the method including inserting at the implantation site an implantable medical device including a substrate coated with P-15 peptide, wherein the implantable medical device is a neurologic device, a vascular device, a cardiovascular device, an oral device, an ocular device, a nasal device, a urogenital device, a gastrointestinal device, a biliary device, an aural device, a subcutaneous device, a plastic surgical device, a general surgical device, or a prosthetic device. In some embodiments, the implantable medical device is a membrane, a mesh, a sling, a tissue anchor, a tissue expander, a suture, or a gel. In some embodiments, the implantable medical device is an extra-cellular matrix (ECM). In some embodiments, the implantable medical device is a tissue. In some embodiments, the implantable medical device is a transplant. In some embodiments, the implantable medical device is a dialysis device. In particular embodiments, the neurologic device is an electrode, pulse generator, or neurovascular catheter; wherein the vascular device is a vascular stent or a vascular graft; wherein the cardiovascular device is a pacemaker, a defibrillator, a coronary stent, a cardiovascular catheter, or a heart valve, optionally wherein the heart valve is a tricuspid valve, a pulmonary valve, a mitral valve, or an aortic valve; wherein the oral device is a tracheostomy tube; wherein the ocular device is an intraocular lens, intrastromal corneal ring segment (ICRS), or ophthalmic catheter; wherein the nasal device is a nasal stent; wherein the urogenital device is a mesh, a contraceptive implant, a hernia mesh, a pelvic mesh, a urinary stent, an artificial urinary sphincter, or a urological catheter, optionally wherein the contraceptive implant is an intrauterine device (IUD) or a birth control implant; wherein the gastrointestinal device is a staple, balloon, sleeve, band, a gastric stimulator, or gastrointestinal catheter, optionally wherein the band is a LINX device; wherein the biliary device is a biliary stent; wherein the aural device is a cochlear implant or ear tube; wherein the subcutaneous device is a drug delivery needle or glucose sensor; wherein the prosthetic device is a prosthetic eye, breast implant, a nose prosthesis, a penile implant, or cosmetic implant; or wherein the breast implant is saline breast implant or a silicone breast implant.

In an embodiment of any of the methods of the invention, the implantable medical device includes a polyarylether ketone (PAEK) (e.g., polyether-ether-ketone (PEEK), polyetherketoneketone (PEKK), or poly(aryl-ether-ketone-ether-ketoneketone (PEKEKK)).

In an embodiment of any of the methods of the invention, the implantation of the implantable medical device reduces local inflammation at the site (e.g., in comparison to implantation of an otherwise identical implantable medical device without administering P-15 coated substrate at the site).

In another embodiment of any of the methods of the invention, the implantation of the implantable medical device reduces local fibrosis at the site (e.g., in comparison to implantation of an otherwise identical implantable medical device without administering P-15 coated substrate at the site).

In various embodiments of any of the methods of the invention, the method optionally includes one or more of the following features: (i) the substrate is not a calcified substrate; (ii) the implantation site does not include bone tissue; (iii) the implantation site is a soft tissue; and/or (iv) the implantable medical device includes a biodegradable polymer.

In a related aspect, the invention features an implantable medical device including (i) a biodegradable polymer; and (ii) a substrate coated with P-15 peptide. In a related aspect, the invention features an implantable medical device including a biodegradable polymer directly coated with P-15. In a related aspect, the invention features an implantable medical device including a biodegradable polymer including P-15. In some embodiments, the P-15 is incorporated within the biodegradable polymer. In a related aspect, the invention features an implantable medical device including a hydrogel including P-15. For example, the biodegradable polymer can be selected from poly(lactic-co-glycolic acid) (PLGA), poly(lactic acid) (PLA), polycaprolactone (PCL), polylactic acid (PLA), polyglycolic acid (PGA), poly(butylene succinate) (PBS), and sucrose acetate isobutyrate (SAIB).

In a related aspect, the invention features an implantable medical device including (i) a polymer selected from hyaluronic acid and carboxy methyl cellulose; and (ii) a substrate coated with P-15 peptide.

In another aspect, the invention features an implantable medical device designed for implantation into soft tissue and including a substrate coated with P-15 peptide. For example, the implantable medical device can be a neurologic device, a vascular device, a cardiovascular device, an oral device, an ocular device, a nasal device, a urogenital device, a gastrointestinal device, a biliary device, an aural device, a subcutaneous device, or a prosthetic device. In particular embodiments, the neurologic device is an electrode, pulse generator, or neurovascular catheter; wherein the vascular device is a vascular stent; wherein the cardiovascular device is a pacemaker, a defibrillator, a coronary stent, a cardiovascular catheter, or a heart valve, optionally wherein the heart valve is a tricuspid valve, a pulmonary valve, a mitral valve, or an aortic valve; wherein the oral device is a tracheostomy tube; wherein the ocular device is an intraocular lens, intrastromal corneal ring segment (ICRS), or ophthalmic catheter; wherein the nasal device is a nasal stent; wherein the urogenital device is a mesh, a contraceptive implant, a hernia mesh, a pelvic mesh, a urinary stent, an artificial urinary sphincter, or a urological catheter, optionally wherein the contraceptive implant is an intrauterine device (IUD) or a birth control implant; wherein the gastrointestinal device is a staple, balloon, sleeve, band, a gastric stimulator, or gastrointestinal catheter, optionally wherein the band is a LINX device; wherein the biliary device is a biliary stent; wherein the aural device is a cochlear implant or ear tube; wherein the subcutaneous device is a drug delivery device, or glucose sensor; wherein the prosthetic device is a prosthetic eye, breast implant, a nose prosthesis, a penile implant, or cosmetic implant; or wherein the breast implant is saline breast implant or a silicone breast implant.

In an embodiment of any of the implantable devices of the invention, the implantable medical device includes a polyarylether ketone (PAEK) (e.g., polyether-ether-ketone (PEEK), polyetherketoneketone (PEKK), or poly(aryl-ether-ketone-ether-ketoneketone (PEKEKK)).

In a related aspect, the invention features a vascular stent having a surface including a substrate coated with P-15 peptide. In certain embodiments, the stent includes stainless steel, cobalt-chromium alloys, nickel-titanium alloy, platinum, or tantalum alloys coated with P-15 peptide.

In another aspect, the invention features a spinal fusion cage including a substrate coated with P-15 peptide, wherein the substrate is not a calcified substrate. In some embodiments, the spinal fusion cage includes a polyarylether ketone (PAEK) (e.g., polyether-ether-ketone (PEEK), polyetherketoneketone (PEKK), or poly(aryl-ether-ketone-ether-ketoneketone (PEKEKK)).

In some embodiments, the spinal fusion cage includes acellular tissue. In some embodiments, the acellular tissue includes extracellular matrix (ECM), such as Acellular Dermal Matrix (ADM).

In some embodiments, the spinal fusion cage includes a mesh. In some embodiments, the spinal fusion cage includes a collagen mesh.

In some embodiments, the first vertebral body may be implant or bone. In some embodiments, the second vertebral body may be implant or bone.

### Definitions

To facilitate the understanding of this invention, a number of terms are defined below and throughout the disclosure. Unless otherwise defined, all technical and scientific terms used herein have the same meaning as commonly understood by one of ordinary skill in the art to which this invention belongs. The terminology herein is used to describe specific embodiments of the invention, but their usage does not limit the invention, except as outlined in the claims.

Terms such as “a”, “an,” and “the” are not intended to refer to only a singular entity but include the general class of which a specific example may be used for illustration.

As used herein, the term “about” refers to a value that is within 10% above or below the value being described.

As used herein, the term “calcified substrate” refers to a substrate that includes calcium cations and phosphate or hydrogen phosphate anions.

As used herein, the term “P-15 peptide” refers to the peptide of SEQ ID NO. 1.

(SEQ ID NO. 1, “P-15”)
Gly-Thr-Pro-Gly-Pro-Gln-Gly-Ile-Ala-Gly-Gln-Arg-
Gly-Val-Val

As used herein, the term “reduces local inflammation” refers to an observed average reduction in one or more markers of inflammation (e.g., TNFα) at an implantation site following implantation according to the methods of the invention in comparison to implantation of an otherwise identical implantable medical device administered without P-15 peptide. The reduction in local inflammation with the incorporation of P-15 peptide can be measured as described in Example 1.

As used herein, the term “reduces local fibrosis” refers to an observed average reduction in fibrosis at an implantation site following implantation according to the methods of the invention in comparison to implantation of an otherwise identical implantable medical device administered without P-15 peptide.

Other features and advantages of the invention will be apparent from the following Detailed Description, the Drawings, and the claims.

### BRIEF DESCRIPTION OF THE DRAWINGS

FIGS. 1A and 1B are graphs depicting the immunomodulatory effects of P-15 peptide on BMDMs plated on PEEK in culture as described in Example 1. As shown in FIG. 1A, TNFα production was significantly reduced in cells plated with P-15 when compared to cells cultured on PEEK alone. As shown in FIG. 1B, a difference in IL-1β production from MSCs in culture with P-15 when compared to PEEK alone was also observed.

FIGS. 2A and 2B are graphs depicting the ALP activity in BMDMs plated on PEEK and alone in culture as described in Example 1 to measure osteogenic capacity. As shown in FIG. 2A, ALP activity was slightly increased for the P15-L on PEEK but significantly increased for P-15 alone after a 7 day incubation. As shown in FIG. 2B, ALP activity increased for the P15-L on PEEK as compared to the control and PEEK itself following a 28 day incubation.

FIGS. 3A and 3B are images showing devices implanted into the femurs of rabbits having bilateral femoral defects. FIG. 3A shows a control PEEK device. FIG. 3B shows a PEEK devices packed with P-15.

FIG. 4 shows MicroCT data, demonstrating an increase in bone deposition around implants packed with P-15 positioned in the femurs of rabbits having bilateral femoral defects as compared to implants not packed with P-15.

FIG. 5 shows MicroCT methodology for three regions of control and P-15 packed implants positioned in the femurs of rabbits having bilateral femoral defects.

FIG. 6A and FIG. 6B are graphs depicting bone volume at two regions around control and P-15 packed implants in rabbits having bilateral femoral defects as determined by MicroCT. FIG. 6A shows bone volume 1 mm offset from the implant surface. FIG. 6B shows bone volume 0.5 mm offset from the implant surface.

FIGS. 7A-F are graphs depicting bone volume at three peri-implant regions for control and P-15 packed implants in rabbits having bilateral femoral defects as determined by MicroCT. FIG. 7A shows bone volume and FIG. 7B shows the bone density at 1000-500 μm ROI. FIG. 7C shows bone volume and FIG. 7D shows the bone density at 500-136 μm ROI. FIG. 7E shows bone volume and FIG. 7F shows the bone density at 136-0 μm ROI.

FIG. 8 is a graph depicting a statistical analysis of the MicroCT data presented in FIG. 7A, 7C, 7E. The results demonstrated significant differences at the bone-implant interface between the P-15 constructs and the control samples. Samples containing P15 had a significant increase in bone volume in the peri-implant space. The data indicated strong evidence that P-15 promotes bone growth and reduces fibrotic layering, which is known to form around PEEK implants.

FIGS. 9A-E show histological imaging of PEEK devices implanted into rabbits, wherein a fibrotic layer is evident.

FIGS. 10A and 10B show MicroCT imaging of PEEK devices implanted into rabbits, wherein a fibrotic layer is evident.

FIGS. 11A and 11B show MicroCT images depicting PEEK implants in a first region in a first rabbit. FIG. 11A shows a control PEEK implant not having P-15. FIG. 11B shows a PEEK implant packed with P-15.

FIGS. 12A and 12B show MicroCT images depicting PEEK implants in a second region in a first rabbit. FIG. 12A shows a control PEEK implant not having P-15. FIG. 12B shows a PEEK implant packed with P-15.

FIGS. 13A and 13B show MicroCT images depicting PEEK implants in a third region in a first rabbit. FIG. 13A shows a control PEEK implant not having P-15. FIG. 13B shows a PEEK implant packed with P-15.

FIGS. 14A and 14B show MicroCT images depicting PEEK implants in a first region in a second rabbit. FIG. 14A shows a control PEEK implant not having P-15. FIG. 14B shows a PEEK implant packed with P-15.

FIGS. 15A and 15B show MicroCT images depicting PEEK implants in a second region in a second rabbit. FIG. 15A shows a control PEEK implant not having P-15. FIG. 15B shows a PEEK implant packed with P-15.

FIGS. 16A and 16B show MicroCT images depicting PEEK implants in a third region in a second rabbit. FIG. 16A shows a control PEEK implant not having P-15. FIG. 16B shows a PEEK implant packed with P-15.

FIGS. 17A and 17B show MicroCT images depicting PEEK implants in a first region in a third rabbit. FIG. 17A shows a control PEEK implant not having P-15. FIG. 17B shows a PEEK implant packed with P-15.

FIGS. 18A and 18B show MicroCT images depicting PEEK implants in a second region in a third rabbit. FIG. 18A shows a control PEEK implant not having P-15. FIG. 18B shows a PEEK implant packed with P-15.

FIGS. 19A and 19B show MicroCT images depicting PEEK implants in a third region in a third rabbit. FIG. 19A shows a control PEEK implant not having P-15. FIG. 19B shows a PEEK implant packed with P-15.

FIGS. 20A-D are graphs depicting pro-inflammatory cytokine concentration in the femurs of rabbits having control and P-15 packed implants positioned therein at 4 weeks and 8 weeks from a core sample surrounding the implant (FIG. 20A and FIG. 20C) and a graft sample inside the implant window (FIG. 20B and FIG. 20D). FIG. 20A and FIG. 20B show average TNF-α concentration and FIG. 20C and FIG. 20D show average IL-1β concentration. FIG. 20E and FIG. 20F are graphs depicting the change in these cytokines over 8 weeks. FIG. 20E shows the change in concentration of TNF-α and IL-1β for the control implants. FIG. 20F shows the change in concentration of TNF-α and IL-1β for the implants packed with P-15. The results indicate a spike in both TNF-α and IL-1β concentration in the femurs of rabbits having P-15 packed implants, indicating an increase in osteoblast proliferation.

FIG. 21A and FIG. 21B are graphs depicting average IL-6 concentration in the femurs of rabbits having control and P-15 packed implants positioned there at 4 weeks and 8 weeks from a core sample surrounding the implant (FIG. 21A) and a graft sample inside the implant window (FIG. 21B). FIG. 21C shows the change in concentration of IL-6 for the control implants. FIG. 21D shows the change in concentration of TNF-α and IL-6 for the implants packed with P-15. The results demonstrate a spike in IL-6 in the femurs of rabbits having P-15 packed implants.

FIG. 22A and FIG. 22B are graphs depicting average IL-4 concentration in the femurs of rabbits having control and P-15 packed implants positioned there at 4 weeks and 8 weeks from a core sample surrounding the implant (FIG. 22A) and a graft sample inside the implant window (FIG. 22B). The results demonstrate a decrease in IL-4 for both the P-15 packed implants and control implants, with the decrease being more significant in the P-15 packed implants

FIG. 23A and FIG. 23B are graphs depicting average IL-2 concentration in the femurs of rabbits having control and P-15 packed implants positioned there at 4 weeks and 8 weeks from a core sample surrounding the implant (FIG. 23A) and a graft sample inside the implant window (FIG. 23B). The results demonstrate a significant decrease in IL-2 concentration over time for the P-15 packed implants.

FIG. 24 shows a schematic summary of the stages of bone healing and the temporal pattern of the relative immune cells and cytokines/growth factors expression.

### DETAILED DESCRIPTION OF THE INVENTION

Foreign body reaction (FBR) and implant debris-induced bioreactivity/inflammation is mostly a peri-implant phenomenon caused by local innate immune cells (e.g., macrophages) that produce proinflammatory cytokines such as tumor necrosis factor-α, among others.

Fibrosis is essentially disorganized tissue regeneration. It originates from an increase in the production of collagen I and III, fibronectin, and proteoglycans due to TGF-β overproduction from inflammatory FBR progression. These elements combine intra- and intermolecularly, leading to the formation of collagen bundles. In addition, a concurrent decrease in matrix degrading proteases, and an up-regulation of protease inhibitors by TGF-β, leads to an environment where ECM formation dominates. Under the influence of TGF-β and PDGF released from macrophages, fibroblast-like cells differentiate into myofibroblasts and proliferate.

This invention features methods and devices for ameliorating implant-induced inflammation and for reducing the risk of fibrosis. The methods and devices of the invention include a substrate coated with P-15 peptide positioned at the site of implantation.

### Interbody Fusion Cages

Interbody fusion cages are implantable devices placed between the bodies of two adjacent vertebrae, after removing the intervertebral disc that typically occupies this space. The cages may be used to treat a number of diseases or disorders, including degenerative disc disease (DDD), spondylolisthesis, spinal tumors, spinal stenosis, or herniated discs. Interbody fusion cages may be placed in the cervical, lumbar, or thoracic spine.

Interbody fusion cages may be in a shape configured to nest between a first intervertebral disc and a second intervertebral disc. For example, the interbody fusion device may include a first surface to be placed in contact with a first intervertebral disc, and a second surface to be placed in contact with a second intervertebral disc. Interbody fusion cages may be cylindrical, circular, rectangular, substantially flat, amorphous, or in the shape of a human intervertebral disc.

Interbody fusion cages may be made of metal, polymer, ceramic, or a fusion of different materials. The interbody fusion cages may have a hollow center or include orifices, which may be filled with a bone-growth promoting material, such as beta-tricalcium phosphate, external organic bone material, or bone material taken from the patient themselves, such as taken from their hip during the same surgery as the fusion. Interbody fusion cages may be porous, allowing the bone graft to grow from the vertebral body through the cage and into the next vertebral body. Interbody fusion cages may be ridged or include a textured surface.

Interbody fusion cages may include additional hardware, such as pedicle screws and rods, configured to maintain the placement of the interbody fusion cage.
PEEK Interbody Fusion Cages Modified with P-15 Peptide

High performance organic polymers are an emerging alternative to titanium based orthopedic implants. Traditional metallic orthopedic devices risk early implant failure due to their high stiffness, resulting in bone degradation via stress shielding arising from a modulus discontinuity between the implant and the surrounding bone. Polymeric implants provide the prospect of an isoelastic implant-tissue interface, significantly reducing the risk of stress shielding.

The polyaryletherketone (PAEK) polymer family is one such group of emerging alternatives to titanium for the fabrication of orthopedic implants, and includes polyether-ether-ketone (PEEK), polyetherketone (PEK), polyetherketoneketone (PEKK), polyetheretherketoneketone (PEEKK), or poly(aryl-ether-ketone-ether-ketoneketone (PEKEKK).

PEEK, a member of the PAEK polymer family, is a promising candidate for the next generation of orthopedic implant materials because of its bone-like mechanical properties and outstanding thermal and chemical stabilities. PEEK confers stability at high temperatures (exceeding 300° C.), resistance to chemical and radiation damage, compatibility with many reinforcing agents (such as glass and carbon fibers), and greater strength than many metals. In addition, PEEK is radiolucent, allowing surgeons to examine whether bone fills the intervertebral space.

Implants fabricated from PAEK polymers, including PEEK, are often encapsulated by fibrous tissue. The lack of bone integration can ultimately result in implant subsidence and nonunion. Cells on PEEK have been shown to be upregulated mRNAs for chemokine ligand-2, interleukin (IL) 1β, IL6, IL8, and tumor necrosis factor. Cells on PEEK induced the formation of factors strongly associated with cell death/apoptosis, suggesting that that fibrous tissue around PEEK implants arises from an inflammatory environment that favors cell death via apoptosis and necrosis (see, e.g., Olivares-Navarrete et al., Spine, 40(6), 399-404 (2015)).

PEEK (—C₆H₄—OC₆H₄—O—C₆H₄—CO—)ₙ, PEK (—OC₆H₄—CO—C₆H₄—)ₙ, and PEKK (—C₆H₄—OC₆H₄—CO—C₆H₄—CO—)ₙ materials are semi-crystalline polymers. Exemplary PAEK materials that can be used in implants in combination with P-15 peptide as described herein are provided in Table 1. Specific medical grades of PEEK that can be combined with P-15 peptide as described herein are provided in Table 2.

TABLE 1
PAEK Materials Used in Implants

| polymer | Tradename | Manufacturer |
|---|---|---|
| PEEK | OPTIMA ® | Invibio |
| PEEK | VICTREX ® | Victrex |
| PEEK | GATONE ® | Gharda |
| PEEK | KETA-SPIRE ® | Solvay |
| PEEK | ZENIVA ® | Solvay |
| PEEK | VESTAKEEP I ® | Evonik |
| PEK | n/a | Invibio |
| PEKK | PEKK | Dupont |
| PEKK | OXPEKK ® | Oxford Performance Materials |
| PEKEKK | ULTRAPEK ® | BASF |

TABLE 2
Medical Grades of PEEK

| | Manufacturer | | |
|---|---|---|---|
| | General | Medium | Easy |
| | Purpose | Flow Grade | Flow Grade |
| | Invibio product | | |
| | OPTIMA | OPTIMA | OPTIMA |
| | NATURAL ® | LT2 ® | LT3 ® |
| Melt Flow Index | 3.4 | 4.5 | 36.4 |
| Molecular Weight (Mn) | 115,000 | 108,000 | 83,000 |

PAEK materials for use in implants can be processed by injection molding, extrusion, compression molding, and/or powder coating methods.

Implantable grade P-15-modified PAEK polymers can also be incorporated into medical devices as PAEK fibers or PAEK films. Furthermore, the P-15-modified PAEK polymers can be a composite material including, e.g., a radio-opacity agent (e.g., barium sulfate) or reinforcing fibers (e.g., carbon fibers, such as ENDOLIGN®). In particular embodiments the PAEK polymeric implant can have a surface modified to permit covalent attachment of the P-15 peptide, using, e.g., cold plasma, surface etching, or surface grafting methods. In still other embodiments, the surface of the PAEK polymeric implant is coated (using plasma spray methods) with hydroxy apatite particles, which are then subsequently coated with P-15 peptide. In other embodiments, the surface of the PAEK materials can be coated using methods analogous to those described in Examples 4 and 5.

Using the methods of the invention a substrate coated with P-15 peptide is positioned at the site of implantation (e.g., implantation of an interbody fusion cage) to ameliorate implant-induced inflammation and fibrosis.

Biodegradable Polymeric Implants Modified with P-15 Peptide

Despite numerous beneficial attributes, biodegradable polyesters (ie, poly(lactide) (PLA), poly(glycolide) (PGA), poly(lactide-co-glycolide) (PLGA)) have not yet been adopted globally in clinical settings. FBRs caused by acidic by-products of the implant and the variable tissue response to degradation rates have been well documented. For instance, as PLGA degrades, lactic acid and glycolic acid monomers are released in the surrounding tissue. The resulting acidic environment has a profound effect on the cytokine profiles of inflammatory cells surrounding the implants. It has been demonstrated that the decrease in pH alters the amount of vascularization post implantation. In addition, polymeric implants with fast degradation times can also alter the amount of blood vessel formation and implant integration.

Using the methods of the invention a substrate coated with P-15 peptide is positioned at the site of implantation to ameliorate implant-induced inflammation resulting from changes in the local pH with in vivo degradation of the biodegradable polymer.

The biodegradable polymers can be coated with P-15 peptide using, e.g., methods analogous to those described in Examples 4 and 5.

Vascular Stents Modified with P-15 Peptide

Since the first reports of successful angioplasty of human coronary atherosclerotic lesions, restenosis has been encountered as a significant limitation to the long-term efficacy of the procedure. Subsequent studies have supported a critical role for inflammatory cells in the restenotic process. A chronic indwelling stent has a profound effect on the inflammatory response, and the risk of restenosis in the patients who receive them.

Using the methods of the invention a vascular stent is coated with or containing P-15 peptide to ameliorate implant-induced inflammation and reduce the risk of restenosis post-implantation.

The biodegradable polymers may be coated with or admixed with P-15 peptide using, e.g., methods analogues to those described in Examples 3-6.

### EXAMPLES

The following examples are put forth to provide those of ordinary skill in the art with a description of how the devices and methods described herein may be used, made, and evaluated, and are intended to be purely exemplary of the invention and are not intended to limit the scope of what the inventors regard as their invention.

### Example 1. In Vitro Immune Response of PEEK Devices with and without P-15 Peptides

The study of the relationship between the musculoskeletal system and the immune system has become an increasingly important consideration in biomaterials research. In vitro investigation of the immune response, provides insight on the efficacy of current design materials in spinal implant devices and also a potential clinical understanding of complications. With bone grafting materials, the probability of arthrodesis and bone apposition increases due to the selected materials. The potential for adverse events including a fibrotic response presents challenges that should be addressed. When osteobiologics are considered, studies involving the pro-inflammatory cytokine response informs on the mechanism of fibrous development pathway which has been anecdotally reported with PEEK spinal interbody implants. The in vitro cell studies in combination with larger animal studies help understand factors associated with bone grafting materials. The combination of results from three separate kinds of studies would potentially weave a better understanding of PEEK fibrous encapsulation around retrieved implanted PEEK devices. It was hypothesized that IL-1β, IL-4, IL-6, and TNFα expression, would be different for PEEK and by extension PEEK devices in the presence of the osteobiologic containing P-15 peptide.

The purpose of this study was to compare the in vitro pro-inflammatory cytokine response of PEEK as an implant material with and without P-15. Specifically, the cytokine response measured in culture via ELISA techniques with bone derived MSCs informs on the bone implant interface.

Cell culture studies measuring the pro inflammatory response of PEEK substrates with and without P-15 were assessed. Human MSCs (MSC) were cultured on PEEK samples with and without P-15. The expression of key cytokines were quantified from these cultures including IL1-β, IL-4 and TNF. Cytokines IL-4 and TNF were selected due to their activity in the macrophage polarization process; IL-4 and TNF are known to induce polarization towards M2 and M1 phenotypes respectively. IL-1β was quantified as this cytokine has been directly linked to the formation of fibrotic tissue surrounding implants. Quantification of ALP (alkaline phosphatase) levels were also studied in order to assess the osteogenic capacity of cultures supplemented with P-15. Traditionally, MSC's grown on smooth PEEK do not typically express high levels of ALP in culture. Furthermore, fluorescent imaging was used to visualize cellular morphology and cellular density in both cohorts. Finally, the RAW 264.7 macrophage like cell line were used to conduct macrophage polarization assays in order to provide a predictor of in vivo results comparing implanted PEEK dowels with and without P-15 on inorganic particles inside were assessed on MicroCT for bone ongrowth and ingrowth.

Bone marrow derived MSCs (ATCC, PCS-500-012; BMDMs) were passaged following standard techniques in MSC Basal media (ATCC, PCS-500-030). Cells were seeded at a density of 2×10\hat{ }4 cells/mL. Cell viability was assessed after 7 days in culture using a cell titer Glo Assay (Promega). Cytokine production was analyzed using ELISAs on cell conditioned media or cell lysate. The culture plate was incubated for 1, 4, 7, or 14 days at 37 C. IL-1B (RAB0273) and TNFα (RAB1089) sandwich ELISA kits were used for analysis of cell conditioned media and lysate, respectively. Cell lysates were collected in a 1×RIPA buffer. ELISA was conducted following the recommended standard protocol. Statistical analysis was conducted using a one-way ANOVA with multiple comparisons.

BMDMs were cultured for 7 or 28 days on TCPS, in PEEK cups with or without P15-L, or on P15-L alone. At 7 days, cells were lysed with RIPA buffer. At 28 days, BMDM conditioned media was collected and both samples were assessed for ALP activity. A fluorometric ALP assays (ab83371) was conducted following manufacturer protocols. The activity of ALP on 4-methylumbelliferyl phosphate disodium salt (MUP) results in the production of a fluorescent byproduct that was analyzed on the BioTek Cytation (Ex/Em: 360 nm/440 nm). All statistical data analyses was performed using GraphPad Prism (GraphPad Software, San Diego, CA) with a significance threshold of p<0.05.

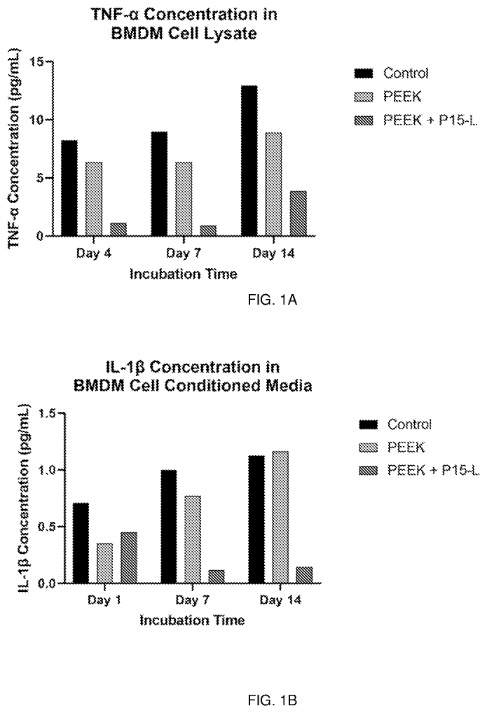

The data shows P-15 has potential immunomodulatory effects on MSCs plated on PEEK in culture. Cells plated on tissue culture polystyrene, PEEK or PEEK with additional P-15 show no significant difference in cell viability following a week in culture. When observing inflammatory cytokine production, the ELISA data showed a significant decrease in TNFα (Table 3; FIG. 1A) and IL-1β (Table 4; FIG. 1B). TNFα production was significantly reduced in cells plated with P-15 after 4, 7 and 14 days in culture when compared to cells cultured on PEEK alone (PEEK+P-15; PEEK (po/mL). We also observed a difference in IL-1β production from MSCs in culture with P-15 when compared to PEEK alone on Day 7 and a significant difference by Day 14.

TABLE 3
TNF-α concentration in the cell lysate from BMDM incubated on TCPS, PEEK, and
PEEK + P15-L for 1, 7, or 14 days. Data represents three technical replicates.
TNF-α ELISA Data

| Day 4 | | | Day 7 | | | Day 14 | | |
|---|---|---|---|---|---|---|---|---|
| | | PEEK + | | | PEEK+ | | | PEEK + |
| Control | PEEK | P15-L | Control | PEEK | P15-L | Control | PEEK | P15-L |

Raw Data

| | | | | | | | | |
|---|---|---|---|---|---|---|---|---|
| 0.133 | 0.094 | 0.017 | 0.1495 | 0.076 | 0.012 | 0.247 | 0.2285 | 0.066 |
| 0.15 | 0.1075 | 0.0135 | 0.1535 | 0.115 | 0.009 | 0.2235 | 0.1895 | 0.046 |
| 0.1375 | 0.1125 | 0.013 | 0.1625 | 0.1235 | 0.0125 | 0.2355 | 0.052 | 0.0655 |

LOG calculation

| | | | | | | | | |
|---|---|---|---|---|---|---|---|---|
| −2.01740 | −2.36446 | −4.07454 | −1.90046 | −2.57702 | −4.42285 | −1.39837 | −1.47622 | −2.7181 |
| −1.89711 | −2.23026 | −4.30506 | −1.87405 | −2.16282 | −4.71053 | −1.49834 | −1.66337 | −3.07911 |
| −1.98413 | −2.18480 | −4.34280 | −1.81708 | −2.09151 | −4.38203 | −1.44604 | −2.95651 | −2.72571 |

Trend line Calculation (pg/mL)

| | | | | | | | | |
|---|---|---|---|---|---|---|---|---|
| 7.852424 | 5.797959 | 1.300749 | 8.69746 | 4.814986 | 0.959378 | 13.48861 | 12.60137 | 4.256445 |
| 8.722877 | 6.519449 | 1.063398 | 8.900501 | 6.915268 | 0.7461 | 12.36004 | 10.7 | 3.104704 |
| 8.084135 | 6.783700 | 1.028895 | 9.354938 | 7.359956 | 0.994224 | 12.93811 | 3.455853 | 4.228249 |

Average TNF-α Concentration (pg/mL)

| | | | | | | | | |
|---|---|---|---|---|---|---|---|---|
| 8.219812 | 6.367036 | 1.131014 | 8.9843 | 6.363403 | 0.899901 | 12.92892 | 8.919073 | 3.863133 |

TABLE 4
IL-1β concentration in the conditioned media from BMDM incubated on TCPS, PEEK, and
PEEK + P15-L for 1, 7, or 14 days. Data represents three technical replicates.
IL-1β ELISA Data

| Day 4 | | | Day 7 | | | Day 14 | | |
|---|---|---|---|---|---|---|---|---|
| | | PEEK + | | | PEEK + | | | PEEK + |
| Control | PEEK | P15-L | Control | PEEK | P15-L | Control | PEEK | P15-L |

Raw Data

| | | | | | | | | |
|---|---|---|---|---|---|---|---|---|
| 0.03 | 0.0065 | 0.032 | 0.0415 | 0.002 | 0.0075 | 0.052 | 0.044 | 0.008 |
| 0.0255 | 0.0005 | 0.009 | 0.0395 | 0.045 | 0.003 | 0.0405 | 0.0385 | 0.0045 |
| 0.023 | 0.0315 | 0.008 | 0.003 | 0.041 | 0.001 | 0.037 | 0.0515 | 0.002 |

LOG calculation

| | | | | | | | | |
|---|---|---|---|---|---|---|---|---|
| −3.50656 | −5.03595 | −3.44202 | −3.18206 | −6.21461 | −4.89285 | −2.95651 | −3.12357 | −4.82831 |
| −3.66908 | −7.6009 | −4.71053 | −3.23145 | −3.10109 | −5.80914 | −3.20645 | −3.2571 | −5.40368 |
| −3.77226 | −3.45777 | −4.82831 | −3.41125 | −3.19418 | −6.90776 | −3.29684 | −2.96617 | −6.21461 |

Trend line Calculation (pg/mL)

| | | | | | | | | |
|---|---|---|---|---|---|---|---|---|
| 0.804562 | 0.194413 | 0.854258 | 1.087515 | 0.065065 | 0.222044 | 1.340923 | 1.148227 | 0.23576 |
| 0.691851 | 0.017957 | 0.263011 | 1.038757 | 1.172442 | 0.094816 | 1.063157 | 1.014313 | 0.13817 |
| 0.628632 | 0.841856 | 0.23576 | 0.879023 | 1.075341 | 0.34181 | 0.977561 | 1.328945 | 0.065065 |

Average IL-1β Concentration (pg/mL)

| | | | | | | | | |
|---|---|---|---|---|---|---|---|---|
| 0.708348 | 0.351408 | 0.45101 | 1.001765 | 0.77095 | 0.117014 | 1.127214 | 1.163829 | 0.146332 |

TABLE 5
ALP activity measured by dephosphorylation of fluorescently tagged MUP in BMDM
cell lysate following a 7 day incubation. Data represents three technical replicates.
7 Day ALP Activity

| | | | | | | Dilution and | | |
|---|---|---|---|---|---|---|---|---|
| RAW data | | | Trend line Calculation | | | Dephosphorylated MUP (mU/min) | | |
| | PEEK + | | | PEEK + | | | PEEK + | |
| PEEK | P15-L | P15-L | PEEK | P15-L | P15-L | PEEK | P15-L | P15-L |
| 5.060329 | 7.02776 | 8.08618 | 3.066866 | 4.259248 | 4.900715 | 6.133732 | 8.518497 | 9.801431 |
| 5.371703 | 5.843786 | 8.078647 | 3.255577 | 3.541688 | 4.89615 | 6.511155 | 7.083377 | 9.792299 |
| 7.646741 | 6.499178 | 11.97961 | 4.634389 | 3.938896 | 7.26037 | 9.268777 | 7.877791 | 14.52074 |

Average dephosphorylation MUP (mU/min)

| Day 4 | Day 7 | Day 14 |
|---|---|---|
| PEEK | PEEK + P15-L | P15-L |

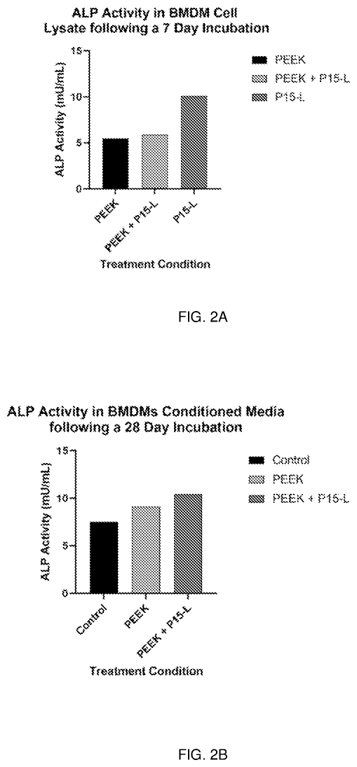

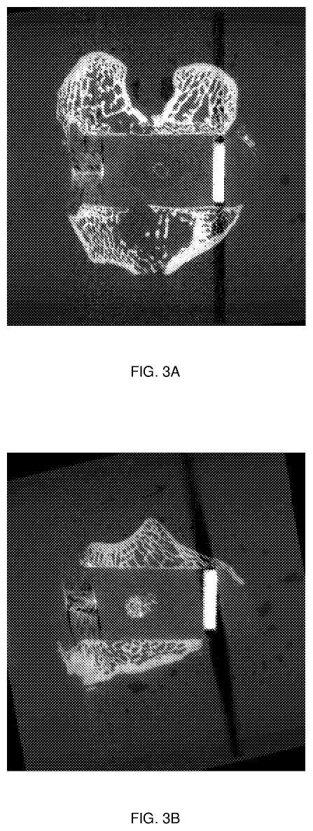

ALP is an enzyme upregulated during osteoblast differentiation. There is an increase in ALP activity in BMDM lysate or cell media at both 7 days and 28 days in cells incubated on P15-L alone (Tables 5 and 6). When cells were incubated on PEEK dishes filled with P15-L we observed a slight increase in ALP activity, but cells incubated on P15-L alone increased the production of ALP (FIGS. 2A and 2B).

To evaluate osteogenic differentiation capacity of P15-L in vitro, alkaline phosphatase activity was evaluated in BMDMs incubated in osteogenic media. Cells incubated on P15-L alone without PEEK show an increase in the ALP activity when compared to PEEK alone or PEEK cups filled with P15-L after 7 days in culture. After 28 days, there was an increase in the ALP activity in the cells incubated on PEEK with P15-L when compared to control or PEEK alone, but this data was insignificant.

Biomedical implant development is an avid area of research due to the ongoing necessity of strong, durable, immunomodulatory materials in several surgical procedures. PEEK has recently been developed and used in the field of orthopedics. Although PEEK is a ubiquitous orthopedic spinal implant material, the inert, hydrophobic surface disfavors cell adhesion, attachment, and growth, which has the potential to lead to a persistent inflammatory response, fibrosis, and implant failure. The results suggest the addition of a P-15 as bone grafting material has a significant immunomodulation effect on osteoblast like cells in the presence of PEEK in culture. We show no significant differences in cell viability of human MSCs cultured with addition of P-15. Importantly, we observed a significant decrease in the expression of inflammatory cytokines after 7 or 14 days in culture with addition of P-15. The data suggest P-15 filled PEEK implants could be advantageous for the suppression of an inflammatory response, leading to better bone formation adjacent to the PEEK.

TABLE 6
ALP activity measured by dephosphorylation of fluorescently tagged MUP in BMDM cell
conditioned media following a 28 day incubation. Data represents three technical replicates.
28 Day ALP Activity

| | | | | | | Dilution and | | |
|---|---|---|---|---|---|---|---|---|
| RAW data | | | Trend line Calculation | | | Dephosphorylated MUP (mU/min) | | |
| | | PEEK + | | | PEEK + | | | PEEK + |
| TCPS | PEEK | P15-L | TCPS | PEEK | P15-L | TCPS | PEEK | P15-L |
| 6674 | 7886 | 7795 | 9.257599 | 10.8672 | 10.75295 | 11.22133 | 13.17237 | 13.03388 |
| 4582 | 6066 | 8377 | 6.718897 | 8.582119 | 11.48367 | 8.144118 | 10.40257 | 13.9196 |
| 6290 | 7726 | 8981 | 8.86336 | 10.66632 | 12.24202 | 10.74347 | 12.92887 | 14.83881 |

Average dephosphorylation MUP (mU/min)

| Day 4 | Day 7 | Day 14 |
|---|---|---|
| TCPS | PEEK | PEEK + P15-L |
| 7.527229 | 9.12595 | 10.44807 |

### Example 2. In Vivo Immune Response of Devices with and without P-15 Peptides

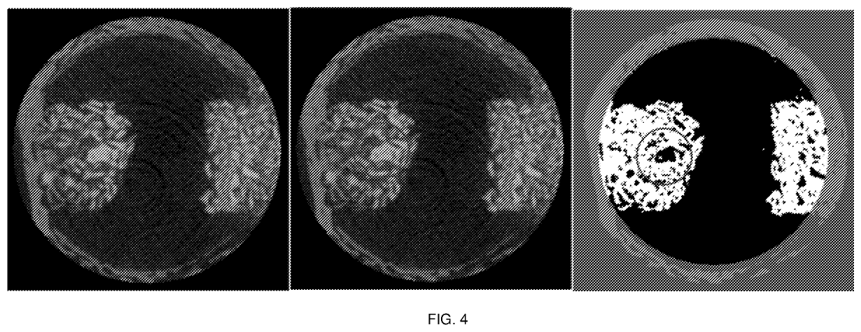

The immune response of P-15 peptides was studied in vivo in rabbits. Seventeen rabbits having bilateral femoral defects were implanted with devices (FIG. 3A-B) loaded with P-15. FIG. 3A shows an implanted PEEK control device. FIG. 3B shows an implanted PEEK device packed with P-15. Three rabbits were euthanized in the immediate post-operative period. Seven rabbits survived four weeks and seven rabbits survived eight weeks.

Cytokine analysis was completed using R&D Systems Rabbit Duo-Set ELISA kits for IL-2, IL-4, IL-6, TNF-alpha, and IL-1β. Manufacturers protocols were followed for each cytokine analyzed.

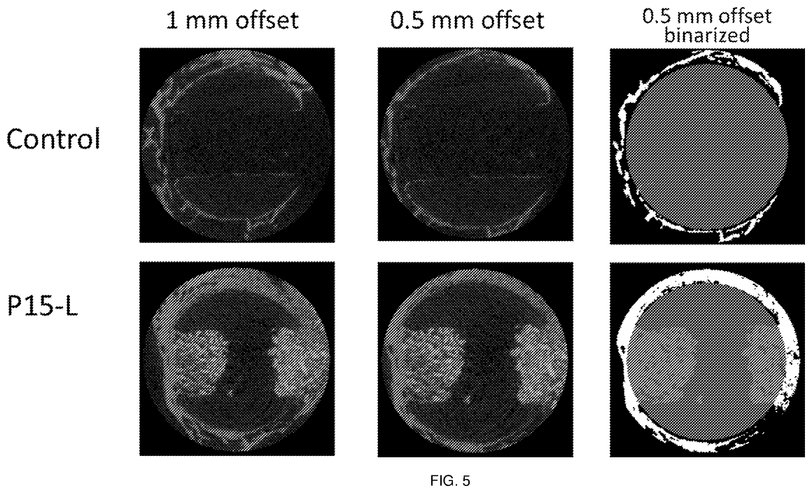

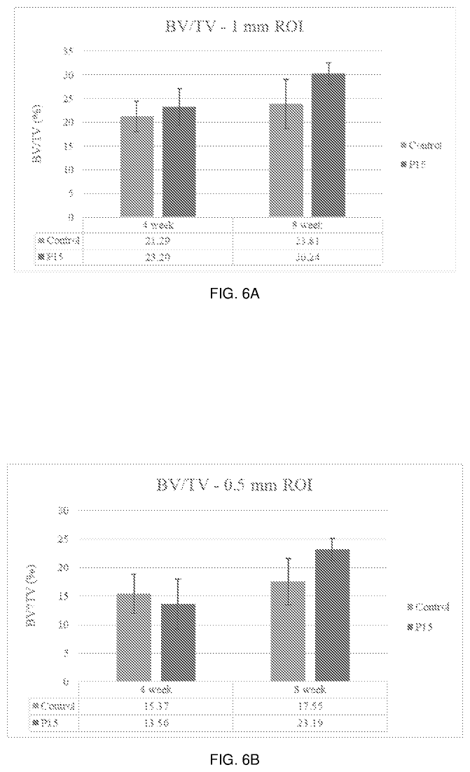

MicroCT analysis was conducted to the study the effect of P-15 on bone deposition (FIG. 4). Three rabbits underwent MicroCT analysis at the 4- and 8-week time point. Preliminary analysis was restricted to the central region of the implant which reduced the impact of implant placement and image artifacts. Quantification was performed in 3 regions (FIG. 5): 1 mm offset from implant surface (8000 μm ROI), 0.5 mm offset from implant surface (7000 μm ROI), and in the graft window. The 1 mm offset was intended to correlate with cytokine analysis and provided assessment of bone growth surrounding the central dowel region. The 0.5 mm offset was intended to focus on bone growth in the peri-implant region and illustrated the amount of bone-implant contact. The graft window was intended to illustrate the amount of bone growth into the central graft window. Bone volume fraction (BV/TV) was quantified for the 1 mm offset (FIG. 6A) and the 0.5 mm offset (FIG. 6B), correlating to the percent of the total region of interest occupied by mineralized bone tissue. The MicroCT data for the preliminary analysis demonstrated that the P-15 constructs showed more overall bone deposition around the implant than the control samples. In the control samples there was a lack of mineral deposition, suggesting improper bone repair phase and a lack of bone remodeling phase.

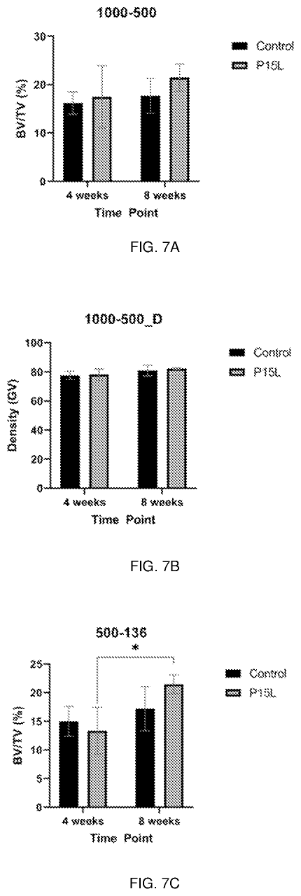

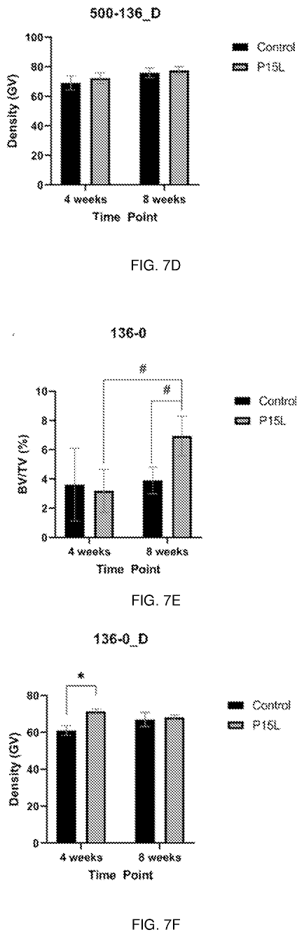

Secondary microCT analysis performed was intended to investigate the influence of P15-L on the surrounding tissues while minimizing imaging artifacts and the influence of residual graft product. For this reason, the analysis was performed in regions of interest surrounding the implant, with the central graft window excluded from all analyses. BV/TV was quantified for the 1000-500 μm ROI (FIG. 7A and FIG. 7B), the 500-136 μm ROI (FIG. 7C and FIG. 7D), and the 136-0 μm ROI (FIG. 7E and FIG. 7F), correlating to the percent of the total region of interest occupied by mineralized bone tissue. The largest of these regions of interest, 1000-500 μm from the implant surface was intended to provide additional context to the immunogenic assessment and give a wholistic understanding of bone growth around PEEK implants (FIG. 7A and FIG. 7B). The second region of interest 500-136 μm provides a more sensitive assessment of the influence of P15L on the surrounding micro-environment (FIG. 7C and FIG. 7D). Results from this ROI showed a significant increase in bone growth from the 4- to 8-week timepoint within the P15L cohort that was not observed in the control cohort. This could indicate a more positive bone growth environment attributed to the presence of the P15-L product. This trend is supported by the results from the smallest ROI. Extending only 136 μm from the implant surface this analysis was intended to focus on bone growth only within the peri-prosthetic region and correlate with the thickness of fibrous capsule formation (FIG. 7E and FIG. 7F). Results within this region again showed a significant increase in bone volume for the P15-L cohort between the 4- and 8-week time points. A significant higher bone volume fraction was also observed in the 8-week P15-L cohort compared to the control cohort. Furthermore, the density of bone observed at the 4-week time point was significantly denser in the P15-L cohort compared to the control cohort. Taken together, these results indicate that P15-L supports faster bone deposition and transition from immature woven bone to mature mineralized tissues.

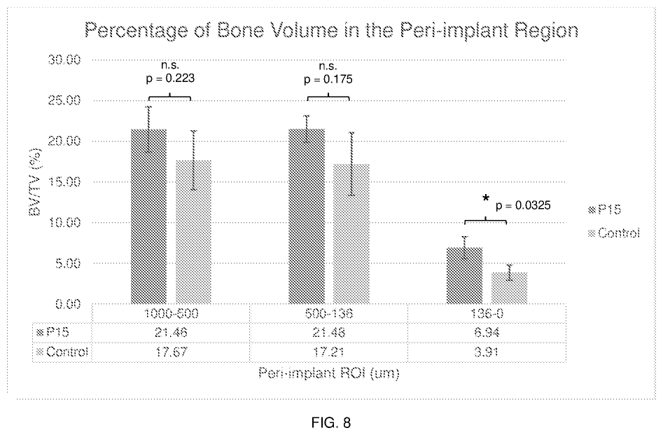

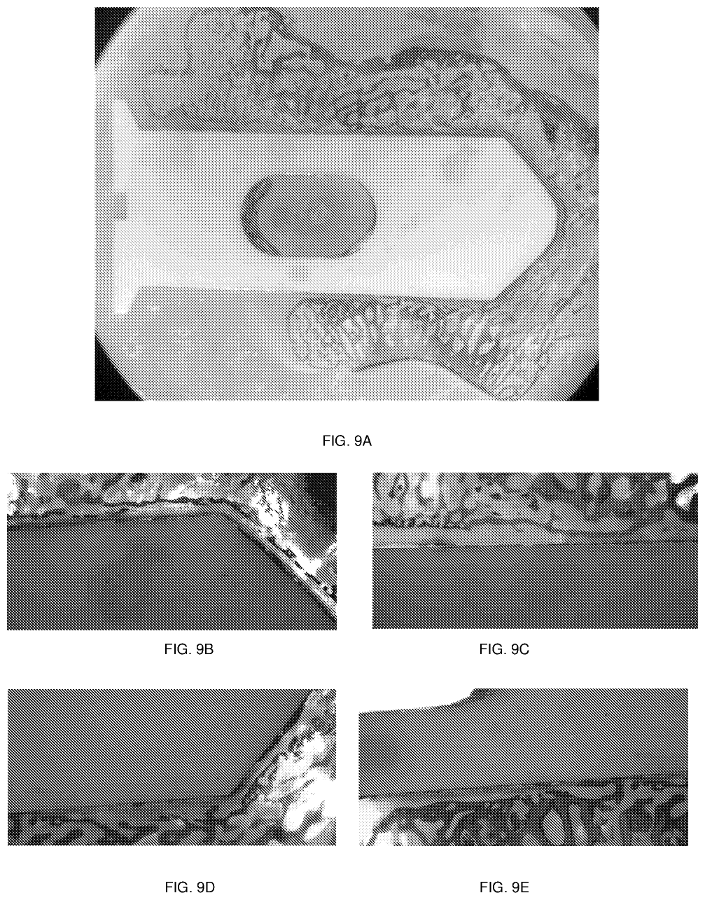

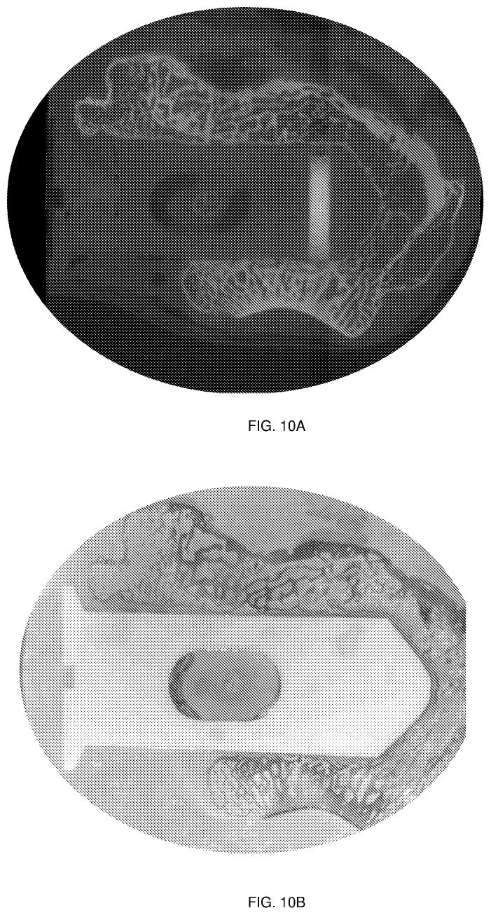

FIG. 8 shows a statistical analysis of the percentage of bone volume in the peri-implant regions at 8 weeks post-operative. Secondary analysis results also indicated significant differences at the bone-implant interface between the P-15 constructs and the control samples. Samples containing P15 had a significant increase in bone volume in the peri-implant space. The observed effect of bone formation on the outside of the implant packed with P-15 was away from the location of the P-15 coated inorganic particles. The data indicated strong evidence that P-15 promotes device ongrowth and reduces fibrotic layering, which is known to form around PEEK implants. FIG. 9A-E shows histological imaging of PEEK devices implanted into rabbits, wherein a fibrotic layer is evident. FIG. 10A-B shows MicroCT imaging of PEEK devices implanted into rabbits, wherein a fibrotic layer is evident.

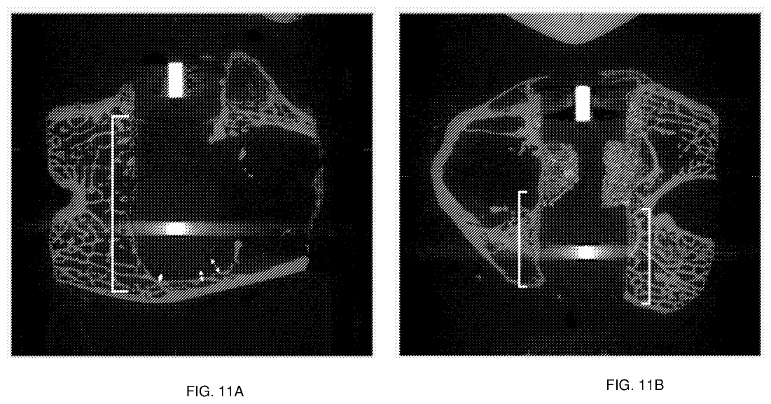

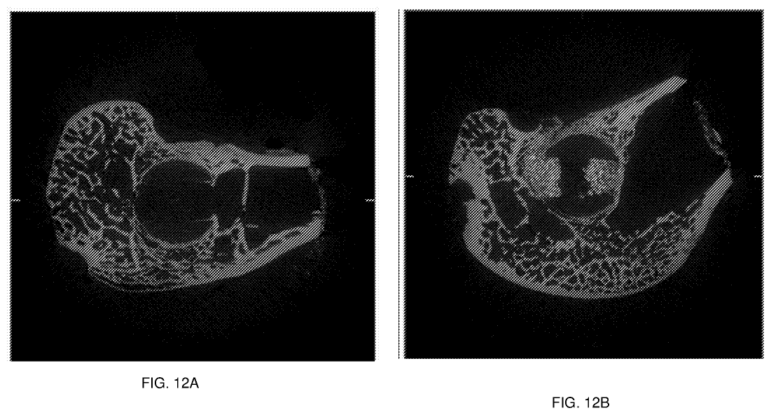

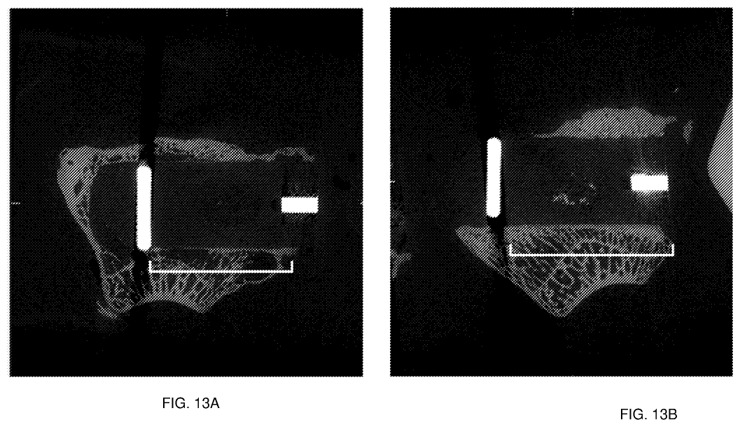

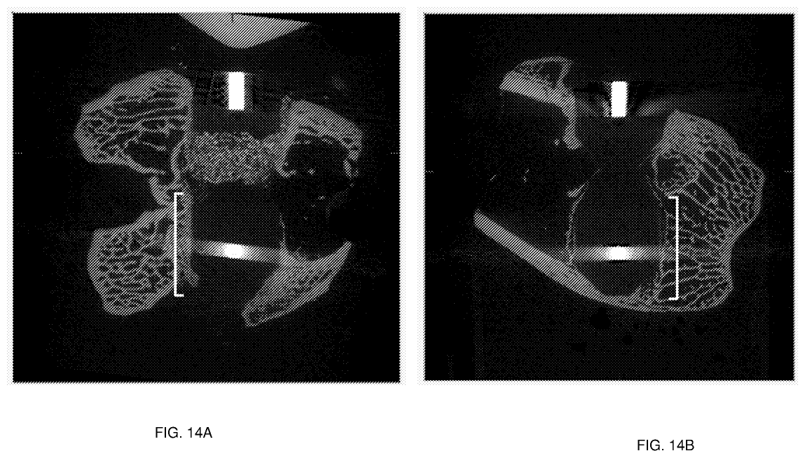

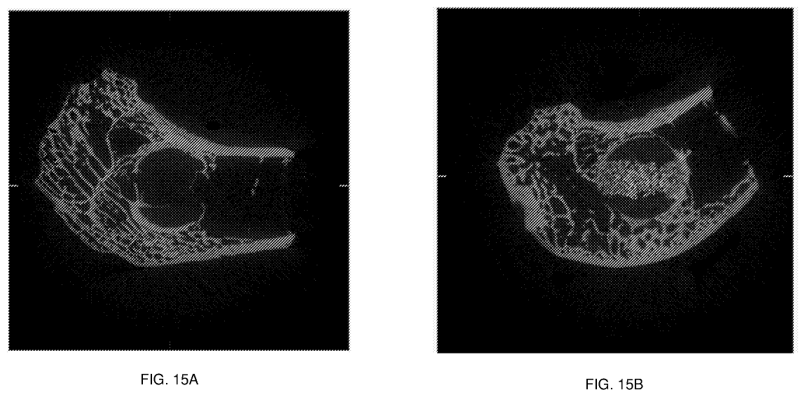

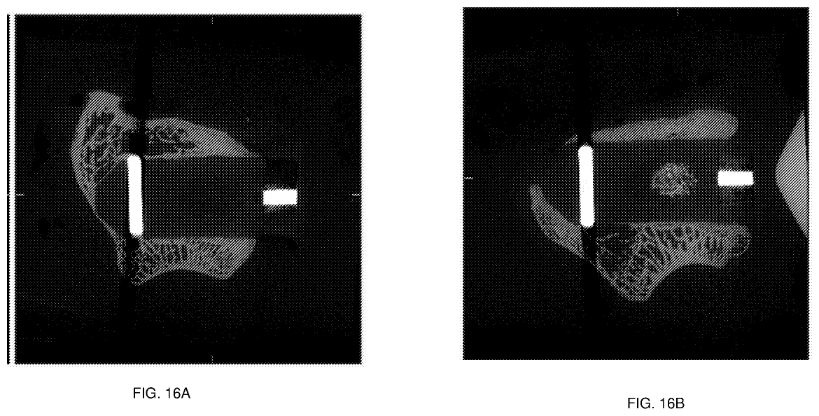

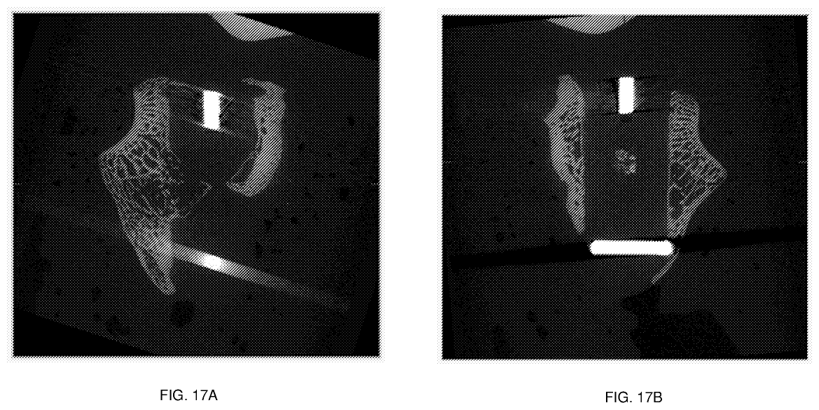

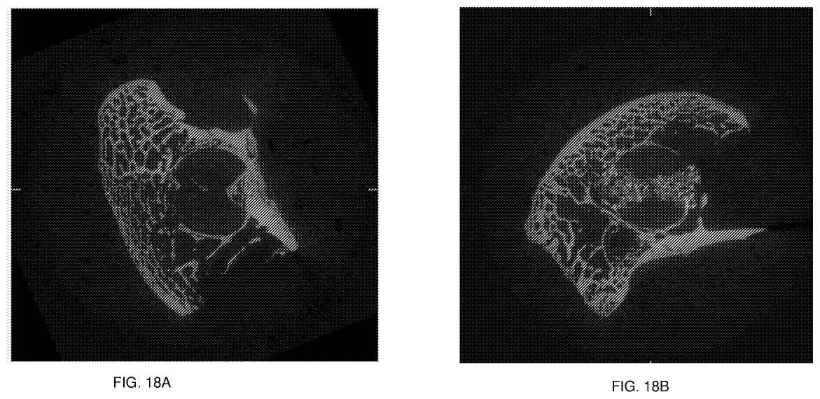

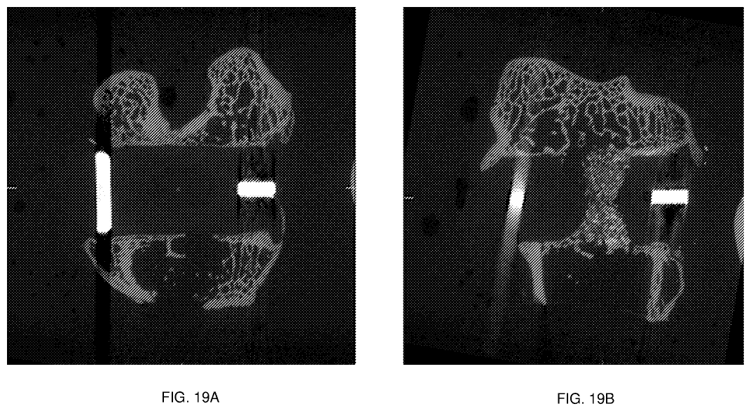

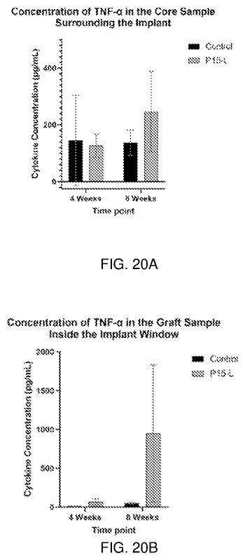

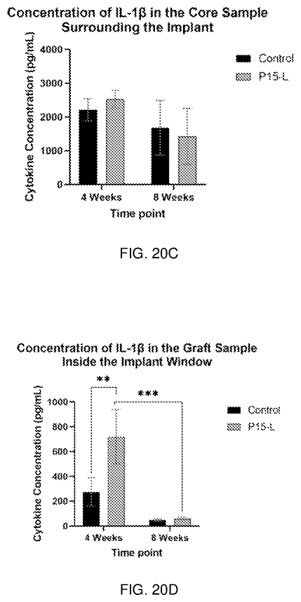

FIG. 11-19 show additional MicroCT images of control devices and devices packed with P-15 implanted into rabbits. FIG. 11A-B show MicroCT images depicting PEEK implants in a first region in a first rabbit. FIG. 11A shows a control PEEK implant not having P-15. FIG. 11B shows a PEEK implant packed with P-15. FIG. 12A-B show MicroCT images depicting PEEK implants in a second region in a first rabbit. FIG. 12A shows a control PEEK implant not having P-15. FIG. 12B shows a PEEK implant packed with P-15. FIG. 13A-B show MicroCT images depicting PEEK implants in a third region in a first rabbit. FIG. 13A shows a control PEEK implant not having P-15. FIG. 13B shows a PEEK implant packed with P-15. FIG. 14A-B show MicroCT images depicting PEEK implants in a first region in a second rabbit. FIG. 14A shows a control PEEK implant not having P-15. FIG. 14B shows a PEEK implant packed with P-15. FIG. 15A-B show MicroCT images depicting PEEK implants in a second region in a second rabbit. FIG. 15A shows a control PEEK implant not having P-15. FIG. 15B shows a PEEK implant packed with P-15. FIG. 16A-B show MicroCT images depicting PEEK implants in a third region in a second rabbit. FIG. 16A shows a control PEEK implant not having P-15. FIG. 16B shows a PEEK implant packed with P-15. FIG. 17A-B show MicroCT images depicting PEEK implants in a first region in a third rabbit. FIG. 17A shows a control PEEK implant not having P-15. FIG. 17B shows a PEEK implant packed with P-15. FIG. 18A-B show MicroCT images depicting PEEK implants in a second region in a third rabbit. FIG. 18A shows a control PEEK implant not having P-15. FIG. 18B shows a PEEK implant packed with P-15. FIG. 19A-B show MicroCT images depicting PEEK implants in a third region in a third rabbit. FIG. 19A shows a control PEEK implant not having P-15. FIG. 19B shows a PEEK implant packed with P-15. The images demonstrate that the devices packed with P-15 show improved bone growth. Brackets indicate areas of visually pronounced differences in bone growth.

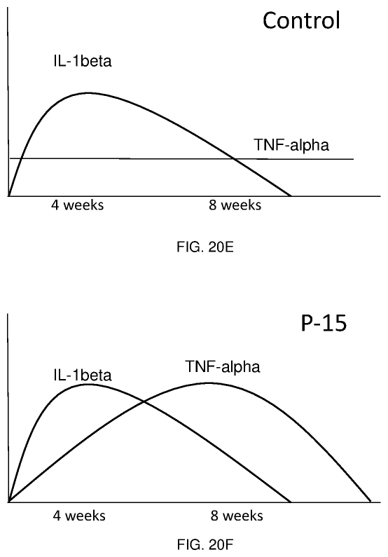

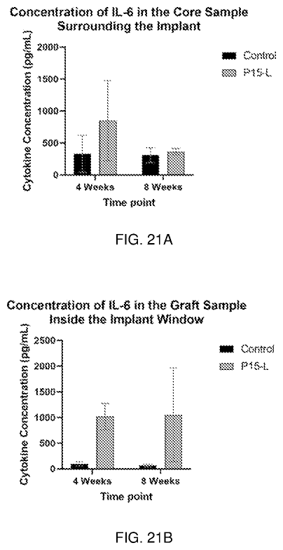

Both pro- and anti-inflammatory cytokines were quantified in the tissue directly surrounding the implant (core) and in the graft window (graft) from both 4- and 8-week cohorts. The pro-inflammatory cytokines evaluated were IL-1β, IL-6 and TNF-α, while IL-4 and IL-2 were evaluated as anti-inflammatory cytokines (FIG. 20-23 and Tables 7-16). A similar trend in concentration is seen in both types of samples from 4- to 8-weeks.

While observing the trends in cytokine expression it became clear that the P15L samples displayed a more active cellular environment, as indicated by higher cytokine expression across all assays. We hypothesize this increase in cytokine expression may indicate a shift from repair to remodeling phase between 4 and 8 weeks. The concentration of majority of the cytokines in the control treatment groups remained unchanged from 4 to 8 weeks, while in the P15-L treatment group, we observed concentration shifts between the time points.

One subject was excluded from the analysis due to exceedingly high cytokine response. Femur samples were snap frozen during necropsy and stored at −80° C. Two samples were isolated for analysis from each femur: the core, the bone surrounding the exterior of the implant, and the graft, the tissue within the central graft window of each implant. Tissue samples were then homogenized using a Fisher Bead Mill. IL-1β and TNF-α are pro-inflammatory cytokines that are necessary for remodeling as part of healing.

In both graft and core samples, there was an increase in the concentration of TNF-α produced in the tissue from 4 to 8 weeks (FIG. 20A, FIG. 20B, Tables 7 and 8). TNF-α is upregulated in the remodeling stage of healing and functions to promote osteoclastogenesis. Persistent and elevated expression of TNF-α levels in tissue can cause damage and reduce bone volume. As indicated by the micro-CT analysis, there is an increase in bone volume in the P15-L samples when compared to control. A decrease in TNF-α would demonstrate an increase in osteoblast proliferation, while an increase in TNF-α would demonstrate a decrease in osteoblast proliferation. Based on the mineral deposition shown in the MicroCT it was hypothesized that there would be a spike in TNF-α, indicating heathy bone healing related to the P-15, which was confirmed in the analysis (FIG. 20A, FIG. 20F). There was no TNF-α spike in the control sample (FIG. 20A, FIG. 20E). The bone volume and the spike in TNF-α at 8 weeks suggests the body adjusting cytokine expression to induce a healthy remodeling phase of healing.

A second inflammatory cytokine involved in late-stage remodeling is IL-1β. The data shows a decrease in IL-1β concentration from 4 to 8 weeks in both the control and P15-L in both core and graft samples (FIG. 20C-F, Tables 9 and 10). There is a significant difference in the concentration of IL-1β in the graft samples at 4 weeks between control and P15-L. There is also a significant decrease in the concentration of IL-1β in the P15-L cohort from 4 to 8 weeks in the graft samples. In fracture models, IL-1β is produced by osteoblasts at 3-weeks post-injury to stimulate bone remodeling. Here, there was a significant difference in the IL-1β concentration between control and P15-L at 4 weeks. This data suggests osteoblasts and other cell types increase the concentration of IL-1β in the bone tissue when P15-L is present when compared to control. In control samples, there is no significant difference in IL-1β between 4 and 8 weeks, however in the presence of P15-L there is a significant decrease in the concentration by 8 weeks, indicating P15-L could be modulating the expression patterns of cytokines to induce healing.

TABLE 7
The concentration of TNF-α in the tissue directly surrounding the implant (core sample)
at 4 and 8 weeks post implantation. The data points indicated with an asterisk have been
removed from each data set. These data points were determined outliers by a Grubbs test.

| TNF-α Core Concentration: 4 Week | | | |
|---|---|---|---|
| | Initial Slurry | | Dilution Correction |
| RAW data | Concentration | Dilution Correction | (From Slurry) |

| Control | P15-L | Control | P15-L | Control | P15-L | Control | P15-L |
|---|---|---|---|---|---|---|---|
| 1.091777177 | 3.416175915 | 44.732 | 50.934 | 1.559682 | 4.880251 | 34.8838405 | 124.28536 |
| 9.983342398 | 2.784123253 | 50.934 | 44.106 | 14.26192 | 3.977319 | 326.7547967 | 87.71181443 |
| 2.372862572 | 5.816138063 | 44.106 | 45.822 | 3.389804 | 8.308769 | 73.22484407 | 170.043105 |
| *11.74681213 | *12.33100763 | *45.822 | *43.203 | *16.78116 | *17.61573 | *316.9206007 | *941.8775939 |

| | |
|---|---|
| Average Concentration | Standard Deviation |

| Control | P15-L | Control | P15-L |
|---|---|---|---|
| 144.9544938 | 127.3467598 | 158.6 | 41.25 |

| TNF-α Core Concentration: 8 Week | | | |
|---|---|---|---|
| | Initial Slurry | | Dilution Correction |
| RAW data | Concentration | Dilution Correction | (From Slurry) |

| Control | P15-L | Control | P15-L | Control | P15-L | Control | P15-L |
|---|---|---|---|---|---|---|---|
| 4.487740175 | 5.054368362 | 29.238 | 45.809 | 6.411057 | 7.220526 | 9.372324803 | 165.3825431 |
| 10.12573131 | 15.67514306 | 45.809 | 18.834 | 14.46533 | 22.39306 | 136.2200168 | 162.5848231 |
| *2.587867298 | *93.29908318 | 18.834 | 14.521 | *3.696953 | *133.2844 | *153.5973181 | *4275.497129 |
| 4.930854619 | 29.60844765 | 14.521 | 32.078 | 7.044078 | 42.29778 | 182.5648923 | 411.21904 |

| | |
|---|---|
| Average Concentration | Standard Deviation |

| Control | P15-L | Control | P15-L |
|---|---|---|---|
| 137.502719 | 246.3954687 | 44.43 | 142.7 |

One-way ANOVA with Multiple Comparison

| TNF-α Core Sample Comparison | P-Value |
|---|---|
| 4 Week Control vs P15-L | 0.6480 |
| 8 Week Control vs P15-L | 0.9981 |
| 4 Week P15-L vs 8 Week P15-L | 0.3341 |
| 4 Week Control vs 8 Week Control | 0.8848 |

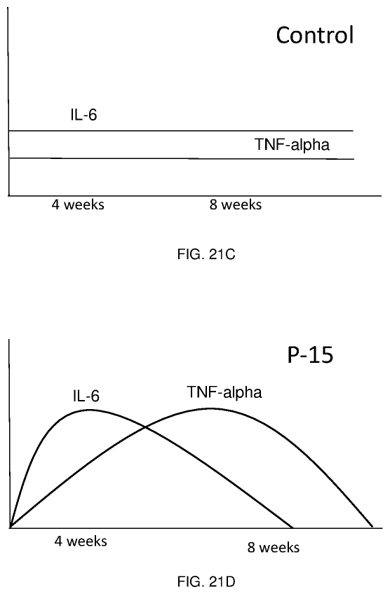

IL-6 is a pro-inflammatory cytokine upregulated in the early inflammatory phase of healing in response to IL-1β stimulation. Our data no significant changes in IL-6 expression at 4 or 8 weeks, in graft or core samples, in control or P15-L samples (FIG. 21A-D, Tables 11 and 12). In future studies, early analysis of IL-6, between days 1-5 would give a better indication of how P15-L may modulate expression.

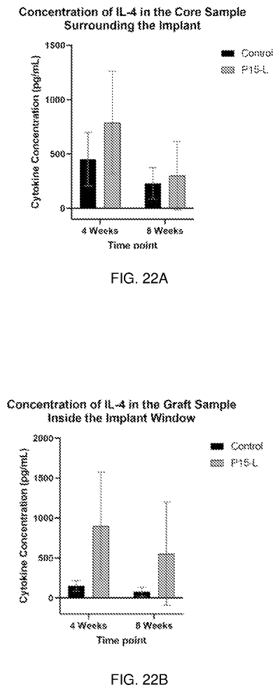

In bone healing, an increase in anti-inflammatory cytokines is necessary to shift from inflammatory to repair phase and reduce the risk of chronic inflammation, and IL-4 is a pro-healing and anti-inflammatory cytokine. IL-4 stimulates M2a macrophage differentiation. M2a macrophages are important in ECM formation and are required in the proliferative phase of wound healing. Based on literature, it was expected that there would be a gradual decrease in anti-inflammatory cytokines as healing progressed. However, the data set indicates no significant differences between the expression of IL-4 at 4 or 8 weeks and between control and P15-L samples (FIG. 22, Tables 13 and 14). There is a decreasing trend in expression of IL-4 from 4 to 8 weeks in the P15-L, which further exemplifies the hypothesized progression from repair to remodeling phase, but this is insignificant.

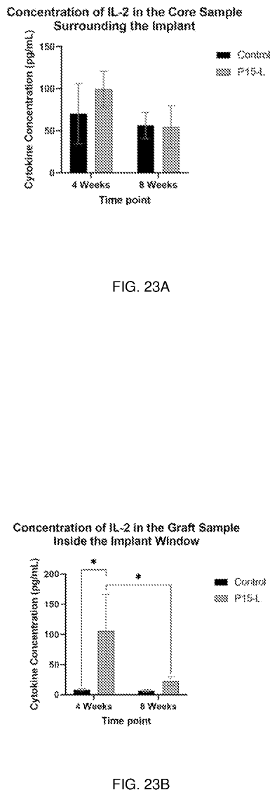

Lastly, the data shows a significant difference in the expression of IL-2 in control verses P15-L samples at 4-weeks in the graft samples (FIG. 23, Tables 15 and 16). There is also a significant decrease in the expression of IL-2 between 4 and 8 weeks for the P15-L samples, whereas in control there is no change. IL-2 is a cytokine with several different functions. Expression of IL-2 promotes local endothelial cell growth and angiogenesis, increasing vascularization to the wound, which is necessary for healing. 12 IL-2 is also required for T-regulatory cell (Tregs) development and function. Tregs are a branch of the adaptive immune system necessary for regulating the immune response. While IL-2 stimulates development of Tregs, Tregs also increase IL-2 receptors and function to sequester additional IL-2, decreasing the concentration in the wound environment. The upregulation in IL-2 in the presence of P15-L at 4 weeks could indicate an increase in Treg development and vascularization to the implant site. The subsequent decrease in IL-2 from 4- to 8-weeks in the P15-L samples could be the result of sequestration by Tregs, allowing to progression into the late remodeling stages of healing.

TABLE 8
The concentration of TNF-α inside the graft window of
the implant (graft sample) at 4 and 8 weeks post implantation.
TNF-α Graft Concentration: 4 Week

| | | Initial Slurry | | | | Dilution Correction | |
|---|---|---|---|---|---|---|---|
| RAW data | | Concentration | | Dilution Correction | | (From Slurry) | |
| Control | P15-L | Control | P15-L | Control | P15-L | Control | P15-L |
| 7.427206204 | 81.70373 | 6.025 | 10.674 | 7.427206204 | 81.70373 | 7.427206204 | 81.70373 |
| 14.19125409 | 27.57993 | 11.47 | 19.811 | 14.19125409 | 27.57993 | 14.19125409 | 27.57993 |
| 13.45076073 | 100.6054 | 6.231 | 10.244 | 13.45076073 | 100.6054 | 13.45076073 | 100.6054 |
| *137.5300639 | *4.001464 | *11.698 | *2.773 | *137.5300639 | *4.001464 | *137.5300639 | *4.001464 |

| Average Concentration | | Standard Deviation | |
|---|---|---|---|
| Control | P15-L | Control | P15-L |
| 69.36531 | 583.1174 | 3.710 | 37.90 |

TNF-α Graft Concentration: 8 Week

| | | Initial Slurry | | | | Dilution Correction | |
|---|---|---|---|---|---|---|---|
| RAW data | | Concentration | | Dilution Correction | | (From Slurry) | |
| Control | P15-L | Control | P15-L | Control | P15-L | Control | P15-L |
| 11.82911362 | 6.55257 | 5.903 | 12.081 | 16.89873 | 9.360814 | 49.87661 | 56.544 |
| 12.86941365 | 156.7462 | 6.703 | 12.081 | 18.38488 | 223.9231 | 61.6191 | 962.7576 |
| *6.320455302 | *785.4589 | *2.702 | *2.992 | *9.029222 | *1122.084 | *12.19848 | *1678.638 |
| 14.39343232 | 157.3217 | 4.156 | 16.248 | 20.56205 | 224.7453 | 42.72793 | 1825.831 |

| Average Concentration | | Standard Deviation | |
|---|---|---|---|
| Control | P15-L | Control | P15-L |
| 51.4072 | 948.3778 | 9.537 | 884.7 |

One-way ANOVA with Multiple Comparison

| TNF-α Core Sample Comparison | P-Value |
|---|---|
| 4 Week Control vs P15-L | 0.9998 |
| 8 Week Control vs P15-L | 0.1438 |
| 4 Week P15-L vs 8 Week P15-L | 0.1550 |
| 4 Week Control vs 8 Week Control | 0.9999 |

The data points indicated with an asterisk have been removed from each data set. These data points were determined outliers by a Grubbs test.

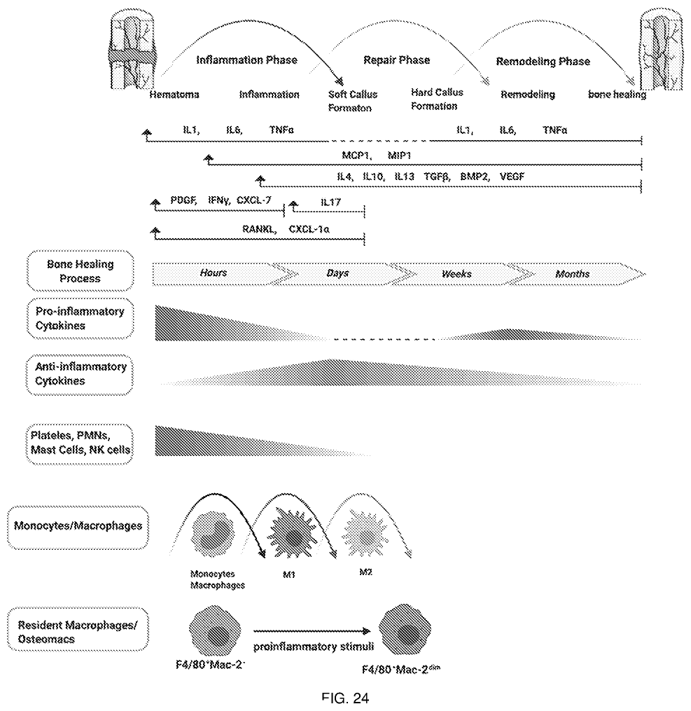

FIG. 24 shows a schematic summary of the stages of bone healing and the temporal pattern of the relative immune cells and cytokines/growth factors expression. Bone healing can be viewed as a three-stage biological phase (inflammation, repair, and remodeling) which can be further divided into six main sub-steps: hematoma, inflammation, soft callus formation, hard callus formation, remodeling, bone healing. After fracture, immune cells including PMNs, NK cells, mast cells, and platelets (platelets are not truly cells as they have no nuclei) are activated in the early stage of the inflammation and the secreted cytokines/chemokines subsequently recruit and activate monocytes/macrophages to further play important roles throughout this process. The pro-inflammatory cytokines including IL1, IL6, TNFα are essential signals during the early stages of bone fracture. In addition, TNFα increases again in the late repair phase, and several pro-inflammatory cytokines (e.g., IL1, IL6, TNFα) are highly expressed in the remodeling phase. The control switch of expression patterns from a pro-inflammatory to an anti-inflammatory response (IL4, IL10, IL 13) in the late stages of inflammation is critical to fracture repair.

TABLE 9
The concentration of IL-1β in the tissue directly surrounding
the implant (core sample) at 4 and 8 weeks post implantation.
IL-1β Core Concentration: 4 Week

| | | Initial Slurry | | | | Dilution Correction | |
|---|---|---|---|---|---|---|---|
| RAW data | | Concentration | | Dilution Correction | | (From Slurry) | |
| Control | P15-L | Control | P15-L | Control | P15-L | Control | P15-L |
| 67.09947 | 69.87334 | 44.732 | 50.934 | 95.85639 | 99.81905 | 2143.924 | 2542.092 |
| 78.62553 | 88.30452 | 50.934 | 44.106 | 112.3222 | 126.1493 | 2573.413 | 2781.971 |
| 62.11119 | 77.22249 | 44.106 | 45.822 | 88.73027 | 110.3178 | 1916.707 | 2257.71 |
| *69.11454 | *74.14834 | *45.822 | *43.203 | *98.73506 | *105.9262 | *1864.661 | *5663.662 |

| | |
|---|---|
| Average Concentration | Standard Deviation |

| Control | P15-L | Control | P15-L |
|---|---|---|---|
| 69.36531 | 583.1174 | 3.710 | 37.90 |

IL-1β Core Concentration: 8 Week

| | | Initial Slurry | | | | Dilution Correction | |
|---|---|---|---|---|---|---|---|
| RAW data | | Concentration | | Dilution Correction | | (From Slurry) | |
| Control | P15-L | Control | P15-L | Control | P15-L | Control | P15-L |
| 70.17327 | 72.04345 | 29.238 | 45.809 | 100.2475 | 102.9192 | 1465.519 | 2357.313 |
| 74.36173 | 73.18769 | 45.809 | 18.834 | 106.231 | 104.5538 | 1000.378 | 759.1131 |
| *69.27635 | *79.31692 | *18.834 | *14.521 | *98.96624 | *113.3099 | *4111.749 | *3634.754 |
| 69.48824 | 83.73063 | 14.521 | 32.078 | 99.26891 | 119.6152 | 2575.802 | 1162.899 |

| | |
|---|---|
| Average Concentration | Standard Deviation |

| Control | P15-L | Control | P15-L |
|---|---|---|---|
| 1679.566 | 1426.442 | 807.8 | 831.1 |

One-way ANOVA with Multiple Comparison

| TNF-α Core Sample Comparison | P-Value |
|---|---|
| 4 Week Control vs P15-L | 0.9583 |
| 8 Week Control vs P15-L | 0.8910 |
| 4 Week P15-L vs 8 Week P15-L | 0.2206 |
| 4 Week Control vs 8 Week Control | 0.7888 |

The data points indicated with an asterisk have been removed from each data set. These data points were determined outliers by a Grubbs test.

TABLE 10
The concentration of IL-1β in the tissue inside the graft window
of the implant (graft sample) at 4 and 8 weeks post implantation.
IL-1β Graft Concentration: 4 Week

| | | Initial Slurry | | | | Dilution Correction | |
|---|---|---|---|---|---|---|---|
| RAW data | | Concentration | | Dilution Correction | | (From Slurry) | |
| Control | P15-L | Control | P15-L | Control | P15-L | Control | P15-L |
| 52.5017 | 70.23941 | 6.025 | 10.674 | 75.00242 | 100.324 | 225.9448 | 535.5253 |
| 49.52164 | 68.04938 | 11.47 | 19.811 | 70.74521 | 97.2134 | 405.7238 | 962.9474 |
| 42.72053 | 91.00511 | 6.231 | 10.244 | 61.02933 | 128.5787 | 190.1369 | 658.5802 |
| *78.97503 | *50.68113 | *11.698 | *2.773 | *112.8215 | *72.40162 | *659.8928 | *100.3848 |

| | |
|---|---|
| Average Concentration | Standard Deviation |

| Control | P15-L | Control | P15-L |
|---|---|---|---|
| 273.935 | 719.018 | 115.5 | 220.0 |

IL-1β Graft Concentration: 8 Week

| | | Initial Slurry | | | | Dilution Correction | |
|---|---|---|---|---|---|---|---|
| RAW data | | Concentration | | Dilution Correction | | (From Slurry) | |
| Control | P15-L | Control | P15-L | Control | P15-L | Control | P15-L |
| 52.50219 | 48.17973 | 5.903 | 12.081 | 52.50219 | 48.17973 | 52.50219 | 48.17973 |
| 51.30825 | 65.54519 | 6.703 | 8.599 | 51.30825 | 65.54519 | 51.30825 | 65.45419 |
| *41.98306 | *77.4904 | *2.702 | *2.992 | *41.98306 | *77.4904 | *41.98306 | *77.4904 |
| 40.31235 | 68.51975 | 4.156 | 16.248 | 40.31235 | 68.51975 | 40.31235 | 68.51975 |

| | |
|---|---|
| Average Concentration | Standard Deviation |

| Control | P15-L | Control | P15-L |
|---|---|---|---|
| 195.566 | 537.855 | 6.720 | 10.99 |

One-way ANOVA with Multiple Comparison

| TNF-α Core Sample Comparison | P-Value |
|---|---|
| 4 Week Control vs P15-L | 0.0093 |
| 8 Week Control vs P15-L | 0.9991 |
| 4 Week P15-L vs 8 Week P15-L | 0.0008 |
| 4 Week Control vs 8 Week Control | 0.2088 |

The data points indicated with an asterisk have been removed from each data set. These data points were determined outliers by a Grubbs test.

TABLE 11
The concentration of IL-6 in the tissue directly surrounding
the implant (core sample) at 4 and 8 weeks post implantation.
IL-6 Core Concentration: 4 Week

| | | Initial Slurry | | | | Dilution Correction | |
|---|---|---|---|---|---|---|---|
| RAW data | | Concentration | | Dilution Correction | | (From Slurry) | |
| Control | P15-L | Control | P15-L | Control | P15-L | Control | P15-L |
| 4.897824 | 15.58118 | 44.732 | 50.934 | 6.996892 | 22.25883 | 156.4925 | 566.8656 |
| 20.34218 | 49.77776 | 50.934 | 44.106 | 29.06026 | 71.11108 | 665.7996 | 1568.213 |
| 5.560805 | 14.23011 | 44.106 | 45.822 | 7.944007 | 20.32783 | 171.6025 | 416.0377 |
| 12.84116 | *17.72437 | *45.822 | *43.203 | *18.34451 | *25.32053 | *346.4453 | *1353.868 |

| | |
|---|---|
| Average Concentration | Standard Deviation |

| Control | P15-L | Control | P15-L |
|---|---|---|---|
| 331.2983 | 850.372 | 289.8 | 626.2 |

IL-6 Core Concentration: 8 Week

| | | Initial Slurry | | | | Dilution Correction | |
|---|---|---|---|---|---|---|---|
| RAW data | | Concentration | | Dilution Correction | | (From Slurry) | |
| Control | P15-L | Control | P15-L | Control | P15-L | Control | P15-L |
| 9.512712 | 11.51808 | 29.238 | 45.809 | 13.58989 | 16.4544 | 198.6662 | 378.8799 |
| 22.36903 | 29.80452 | 45.809 | 18.834 | 31.95576 | 42.57789 | 300.9274 | 309.1368 |
| *6.214213 | *17.74653 | *18.834 | *14.521 | 8.877447 | *25.35219 | *368.8313 | *813.2476 |
| 11.66914 | 29.35051 | 14.521 | 32.078 | 16.6702 | 41.9293 | 432.0499 | 407.6366 |

| | |
|---|---|
| Average Concentration | Standard Deviation |

| Control | P15-L | Control | P15-L |
|---|---|---|---|
| 310.5478 | 364.5511 | 117.0 | 50.39 |

One-way ANOVA with Multiple Comparison

| TNF-α Core Sample Comparison | P-Value |
|---|---|
| 4 Week Control vs P15-L | 0.3657 |
| 8 Week Control vs P15-L | 0.9996 |
| 4 Week P15-L vs 8 Week P15-L | 0.4227 |
| 4 Week Control vs 8 Week Control | 0.9999 |

The data points indicated with an asterisk have been removed from each data set. These data points were determined outliers by a Grubbs test.

TABLE 12
The concentration of IL-6 in the tissue inside the graft window
of the implant (graft sample) at 4 and 8 weeks post implantation.

| IL-6 Graft Concentration: 4 Week | | | |
|---|---|---|---|
| | Initial Slurry | | Dilution Correction |
| RAW data | Concentration | Dilution Correction | (From Slurry) |

| Control | P15-L | Control | P15-L | Control | P15-L | Control | P15-L |
|---|---|---|---|---|---|---|---|
| 17.22674 | 95.95501 | 6.025 | 10.674 | 24.60962 | 137.0786 | 74.13649 | 731.5884 |
| 17.73293 | 85.87924 | 11.47 | 19.811 | 25.33276 | 122.6846 | 145.2834 | 1215.253 |
| 15.60512 | 152.1055 | 6.231 | 10.244 | 22.29303 | 217.2936 | 69.45393 | 1112.978 |
| *175.8345 | *9.157675 | *11.698 | *2.773 | *251.1922 | *13.08239 | *1469.223 | *18.13874 |

| | |
|---|---|
| Average Concentration | Standard Deviation |

| Control | P15-L | Control | P15-L |
|---|---|---|---|
| 96.2913 | 1019.94 | 42.49 | 254.9 |

IL-6 Graft Concentration: 8 Week

| | | Initial Slurry | | | | Dilution Correction | |
|---|---|---|---|---|---|---|---|
| RAW data | | Concentration | | Dilution Correction | | (From Slurry) | |
| Control | P15-L | Control | P15-L | Control | P15-L | Control | P15-L |
| 17.66969 | 10.68764 | 5.903 | 12.081 | 25.24241 | 15.26806 | 74.50298 | 92.2267 |
| 18.9226 | 311.1784 | 6.703 | 8.599 | 27.03229 | 444.5406 | 90.59872 | 1911.302 |
| *14.57812 | *66.52196 | *2.702 | *2.992 | *20.82591 | *95.03137 | *28.13581 | *142.1669 |
| 12.0823 | 99.09295 | 4.156 | 16.248 | 17.26043 | 141.5614 | 35.86717 | 1150.044 |

| | |
|---|---|
| Average Concentration | Standard Deviation |

| Control | P15-L | Control | P15-L |
|---|---|---|---|
| 66.9896 | 1051.19 | 28.13 | 913.6 |

One-way ANOVA with Multiple Comparison

| TNF-α Core Sample Comparison | P-Value |
|---|---|
| 4 Week Control vs P15-L | 0.1661 |
| 8 Week Control vs P15-L | 0.1321 |
| 4 Week P15-L vs 8 Week P15-L | 0.9999 |
| 4 Week Control vs 8 Week Control | 0.9999 |

The data points indicated with an asterisk have been removed from each data set. These data points were determined outliers by a Grubbs test.

TABLE 13
The concentration of IL-4 in the tissue directly surrounding
the implant (core sample) at 4 and 8 weeks post implantation.

| IL-4 Core Concentration: 4 Week | | | |
|---|---|---|---|
| | Initial Slurry | | Dilution Correction |
| RAW data | Concentration | Dilution Correction | (From Slurry) |

| Control | P15-L | Control | P15-L | Control | P15-L | Control | P15-L |
|---|---|---|---|---|---|---|---|
| 8.240169 | 20.59792 | 44.732 | 50.934 | 8.240169 | 20.59792 | 44.732 | 50.934 |
| 22.28445 | 10.7112 | 50.934 | 44.106 | 22.28445 | 10.7112 | 50.934 | 44.106 |
| 11.76633 | 43.78994 | 44.106 | 45.822 | 11.76633 | 43.78994 | 44.106 | 45.822 |
| *33.72021 | *31.63999 | *45.822 | *43.203 | *33.72021 | *31.63999 | *45.822 | *43.203 |

| | |
|---|---|
| Average Concentration | Standard Deviation |

| Control | P15-L | Control | P15-L |
|---|---|---|---|
| 451.9186 | 789.0306 | 245.4 | 472.7 |

| IL-4 Core Concentration: 8 Week | | | |
|---|---|---|---|
| | Initial Slurry | | Dilution Correction |
| RAW data | Concentration | Dilution Correction | (From Slurry) |

| Control | P15-L | Control | P15-L | Control | P15-L | Control | P15-L |
|---|---|---|---|---|---|---|---|
| 4.53026 | 4.001846 | 29.238 | 45.809 | 4.53026 | 4.001846 | 29.238 | 45.809 |
| 15.83849 | 10.48084 | 45.809 | 18.834 | 15.83849 | 10.48084 | 45.809 | 18.834 |
| *8.73678 | *13.23651 | *18.834 | *14.521 | *8.73678 | *13.23651 | *18.834 | *14.521 |
| 10.34344 | 47.7929 | 14.521 | 32.078 | 10.34344 | 47.7929 | 14.521 | 32.078 |

| | |
|---|---|
| Average Concentration | Standard Deviation |

| Control | P15-L | Control | P15-L |
|---|---|---|---|
| 789.0306 | 301.1424 | 144.9 | 314.2 |

One-way ANOVA with Multiple Comparison

| TNF-α Core Sample Comparison | P-Value |
|---|---|
| 4 Week Control vs P15-L | 0.6480 |
| 8 Week Control vs P15-L | 0.9981 |
| 4 Week P15-L vs 8 Week P15-L | 0.3341 |
| 4 Week Control vs 8 Week Control | 0.8848 |

The data points indicated with an asterisk have been removed from each data set. These data points were determined outliers by a Grubbs test.

TABLE 14
The concentration of IL-4 in the tissue inside the graft window
of the implant (graft sample) at 4 and 8 weeks post implantation.
IL-4 Graft Concentration: 4 Week

| | | Initial Slurry | | | | Dilution Correction | |
|---|---|---|---|---|---|---|---|
| RAW data | | Concentration | | Dilution Correction | | (From Slurry) | |
| Control | P15-L | Control | P15-L | Control | P15-L | Control | P15-L |
| 17.61978 | 87.77617 | 6.025 | 10.674 | 17.61978 | 87.77617 | 6.025 | 10.674 |
| 25.26363 | 26.38286 | 11.47 | 19.811 | 25.26363 | 26.38286 | 11.47 | 19.811 |
| 37.68282 | 226.9021 | 6.231 | 10.244 | 37.68282 | 226.9021 | 6.231 | 10.244 |
| *36.58999 | *10.65603 | *11.698 | *2.773 | *26.58999 | *10.65603 | *11.698 | *2.773 |

| | |
|---|---|
| Average Concentration | Standard Deviation |

| Control | P15-L | Control | P15-L |
|---|---|---|---|
| 150.1749 | 900.9472 | 67.31 | 674.0 |

IL-4 Graft Concentration: 8 Week

| | | Initial Slurry | | | | Dilution Correction | |
|---|---|---|---|---|---|---|---|
| RAW data | | Concentration | | Dilution Correction | | (From Slurry) | |
| Control | P15-L | Control | P15-L | Control | P15-L | Control | P15-L |
| 12.55442 | 17.54645 | 5.903 | 12.081 | 12.55442 | 17.54645 | 5.903 | 12.081 |
| 29.643 | 34.41411 | 6.703 | 8.599 | 29.643 | 34.41411 | 6.703 | 8.599 |
| *8.853763 | *87.32398 | *2.702 | *2.992 | *8.853763 | *87.32398 | *2.702 | *2.992 |
| 11.49946 | 112.0362 | 4.156 | 16.248 | 11.49946 | 112.0362 | 4.156 | 16.248 |

| | |
|---|---|
| Average Concentration | Standard Deviation |

| Control | P15-L | Control | P15-L |
|---|---|---|---|
| 76.33952 | 554.35 | 57.58 | 646.7 |

One-way ANOVA with Multiple Comparison

| TNF-α Core Sample Comparison | P-Value |
|---|---|
| 4 Week Control vs P15-L | 0.1661 |
| 8 Week Control vs P15-L | 0.1321 |
| 4 Week P15-L vs 8 Week P15-L | 0.9999 |
| 4 Week Control vs 8 Week Control | 0.9999 |

The data points indicated with an asterisk have been removed from each data set. These data points were determined outliers by a Grubbs test.

TABLE 15
The concentration of IL-2 in the tissue directly surrounding
the implant (core sample) at 4 and 8 weeks post implantation.
IL-2 Core Concentration: 4 Week

| | | Initial Slurry | | | | Dilution Correction | |
|---|---|---|---|---|---|---|---|
| RAW data | | Concentration | | Dilution Correction | | (From Slurry) | |
| Control | P15-L | Control | P15-L | Control | P15-L | Control | P15-L |
| 2.053397 | 3.322687 | 44.732 | 50.934 | 2.053397 | 3.322687 | 44.732 | 50.934 |
| 3.307001 | 2.481793 | 50.934 | 44.106 | 3.307001 | 2.481793 | 50.934 | 44.106 |
| 1.192081 | 3.380081 | 44.106 | 45.822 | 1.192081 | 3.380081 | 44.106 | 45.822 |
| *7.560192 | *4.560935 | *45.822 | *43.203 | *7.560192 | *4.56093 | *45.822 | *43.203 |

| | |
|---|---|
| Average Concentration | Standard Deviation |

| Control | P15-L | Control | P15-L |
|---|---|---|---|
| 70.2113 | 99.29758 | 35.95 | 21.35 |

IL-2 Core Concentration: 8 Week

| | | Initial Slurry | | | | Dilution Correction | |
|---|---|---|---|---|---|---|---|
| RAW data | | Concentration | | Dilution Correction | | (From Slurry) | |
| Control | P15-L | Control | P15-L | Control | P15-L | Control | P15-L |
| 1.847299 | 2.525462 | 29.238 | 45.809 | 1.847299 | 2.525462 | 29.238 | 45.809 |
| 4.566248 | 3.245524 | 45.809 | 18.834 | 4.566248 | 3.245524 | 45.809 | 18.834 |
| *3.15379 | *3.741362 | *18.834 | *14.521 | *3.15379 | *3.741362 | *18.834 | *14.521 |
| 18.853502 | 3.358976 | 14.521 | 32.078 | 1.853502 | 3.358976 | 14.521 | 32.078 |

| | |
|---|---|
| Average Concentration | Standard Deviation |

| Control | P15-L | Control | P15-L |
|---|---|---|---|
| 56.2115 | 54.31645 | 15.69 | 25.37 |

One-way ANOVA with Multiple Comparison

| TNF-α Core Sample Comparison | P-Value |
|---|---|
| 4 Week Control vs P15-L | 0.5961 |
| 8 Week Control vs P15-L | 0.9999 |
| 4 Week P15-L vs 8 Week P15-L | 0.2333 |
| 4 Week Control vs 8 Week Control | 0.9483 |

The data points indicated with an asterisk have been removed from each data set. These data points were determined outliers by a Grubbs test.

TABLE 16
The concentration of IL-2 in the tissue inside the graft window
of the implant (graft sample) at 4 and 8 weeks post implantation.
IL-2 Graft Concentration: 4 Week

| | | Initial Slurry | | | | Dilution Correction | |
|---|---|---|---|---|---|---|---|
| RAW data | | Concentration | | Dilution Correction | | (From Slurry) | |
| Control | P15-L | Control | P15-L | Control | P15-L | Control | P15-L |
| 1.618732 | 16.1418 | 6.025 | 10.674 | 1.618732 | 16.1418 | 6.025 | 10.674 |
| 1.074588 | 2.724978 | 11.47 | 19.811 | 1.074588 | 2.724978 | 11.47 | 19.811 |
| 2.122568 | 21.39336 | 6.231 | 10.244 | 2.122568 | 21.39336 | 6.231 | 10.244 |
| *4.982474 | *1.031474 | *11.698 | *2.773 | *4.982474 | *1.031474 | *11.698 | *2.773 |

| | |
|---|---|
| Average Concentration | Standard Deviation |

| Control | P15-L | Control | P15-L |
|---|---|---|---|
| 8.405739 | 106.0561 | 1.287 | 60.80 |

IL-2 Graft Concentration: 8 Week

| | | Initial Slurry | | | | Dilution Correction | |
|---|---|---|---|---|---|---|---|
| RAW data | | Concentration | | Dilution Correction | | (From Slurry) | |
| Control | P15-L | Control | P15-L | Control | P15-L | Control | P15-L |
| 2.04184 | 4.674467 | 5.903 | 12.081 | 2.04184 | 1.674467 | 5.903 | 12.081 |
| 1.390316 | 4.283474 | 6.703 | 8.299 | 1.390316 | 4.283474 | 6.703 | 8.599 |
| *4.886018 | *5.83331 | *2.702 | *2.992 | *1.886018 | *5.83331 | *2.702 | *2.992 |
| 1.642491 | 2.356059 | 4.156 | 16.248 | 1.642491 | 2.356059 | 4.156 | 16.248 |

| | |
|---|---|
| Average Concentration | Standard Deviation |

| Control | P15-L | Control | P15-L |
|---|---|---|---|
| 6.713919 | 22.70097 | 1.867 | 7.165 |

One-way ANOVA with Multiple Comparison

| TNF-α Core Sample Comparison | P-Value |
|---|---|
| 4 Week Control vs P15-L | 0.0179 |
| 8 Week Control vs P15-L | 0.9554 |
| 4 Week P15-L vs 8 Week P15-L | 0.0407 |
| 4 Week Control vs 8 Week Control | 0.9999 |

The data points indicated with an asterisk have been removed from each data set. These data points were determined outliers by a Grubbs test.

In sum, the P-15 constructs showed increased bioactivity as compared to the control samples, including increased bony deposition, and more pronounced cytokine activity and evaluated the mechanisms underlying the clinical success of P15-L as a bone graft material. This data suggests P15-L may function to modulate cytokine production upon interaction with cells. Changes in expression patterns of both pro-inflammatory and anti-inflammatory cytokines were identified in the presence of P15-L that are not present in control samples. Cell surface interaction with biomaterials induces intracellular signaling cascades, which dictate cell differentiation, proliferation, and extra-cellular signaling. The data presented here indicates that in both the early inflammatory stage of healing and the late remodeling stage P15-L is modulating cytokine production allowing for an increase in bony deposition and overall increased bone healing.

### Example 3: P-15 Coated PEEK Spinal Fusion Cage

As noted above, PEEK is often encapsulated by fibrous tissue. The lack of bone integration can ultimately result in implant subsidence and nonunion.

The surface of a PEEK interbody fusion cage is chemically activated via treatment with a cold plasma (see, e.g., Hubbell et al, Trends Polym. Sci. 2 (1) (1994) 20-25; Lopez et al., Desalination 200 (2006) 503-504; Tang et al., J Biomed Mater Res. 42 (1998) 156-163; and Jha et al., J. Appl. Polym. Sci. 118 (1) (2010)). The activated surface is then reacted with P-15 peptide. This method of treatment can be used to modify different types of surfaces including chemically inert ones, without affecting the bulk chemistry.

The resulting PEEK interbody fusion cage is coated with P-15 peptide. The interbody fusion cage can be implanted into the spine of a subject to replace a damaged spinal disc and promote spinal fusion.

The P-15 coated PEEK interbody fusion cages of the invention can reduce local inflammation post implantation, reducing the risk of fibrous tissue formation, and reducing the risk of implant subsidence and nonunion.

### Example 4: PEEK/ABM-P-15 Composite in a Spinal Fusion Cage

As noted above, PEEK is often encapsulated by fibrous tissue. The lack of bone integration can ultimately result in implant subsidence and nonunion.

A PEEK interbody fusion cage is coated with hydroxyapatite (HA). With regard to the HA coating, the ISO Standard 13779-2 specifies requirements for hydroxyapatite coatings applied to surgical implants, and serves as a guideline for the characterization as well (see ISO Standard 13779-2:2008, Implants for Surgery—Hydroxyapatite—Part 2: Coatings of Hydroxyapatite, International Organization for Standardization, Geneva, Switzerland, 2008). The HA coating can be prepared using plasma spray methods (see, e.g., Paital et al., N. B. Mater. Sci. Eng. R. Rep. 66 (2009) 1-70).

Once the HA-coated PEEK implant is formed, the surface HA is contacted with a solution of P-15 peptide and dried, resulting in a PEEK surface coated with HA which is itself coated with P-15 peptide.

The P-15 coated PEEK interbody fusion cages of the invention can reduce local inflammation post implantation, reducing the risk of fibrous tissue formation, and reducing the risk of implant subsidence and nonunion.

### Example 5: Coating of Titanium with P-15

This Example demonstrates the successful coating of titanium discs with P-15.

Titanium foil discs were obtained from commercial suppliers. Discs were pre-washed with PBS buffer. Discs were submerged in PBS buffer containing P-15. The discs were agitated in the P-15 binding solution overnight. The discs were washed 6 times with PBS and dried overnight in a lyophilizer.

The dried discs were placed in a well of a 24-well plate for a P-15 ELISA test. In parallel, uncoated discs were also test using the ELISA method. The ELISA test is based on the currently validated method to measure the amount of P-15 on anorganic bone mineral. The presence of bound P-15 peptide is demonstrated by an increase in the optical density (OD) of the marker.

Table 17 displays the OD values for the Test and Control discs for the presence of bound P-15 peptide.

TABLE 17
Coating of Titanium Discs with P-15
Titanium Disc

| Un-Coated | P-15 Coated |
|---|---|
| 0.076 +/− 0.014 | 1.069 +/− 0.038 |

The coating technique successfully bound P-15 peptide to the titanium surface.

### Example 6: P-15 Coated Titanium Bare Metal Vascular Stent

The widespread use of coronary stents has fundamentally altered the vascular response to injury by causing a more intense and prolonged inflammatory state. Traditional coronary stent materials include stainless steel (316L), cobalt-chromium alloys, nickel-titanium alloy (Nitinol), platinum, and tantalum alloys, but can also be formed from biodegradable and non-degradable polymers. To address the problem of local inflammation at the site of implantation and the risk of restenosis, vascular stents have been coated with antiproliferative agents (e.g., paclitaxel and rapamycin macrolides. Using the methods of the invention local inflammation can be reduced without antiproliferative agents by coating a surface of the vascular stent with P-15 peptide prior to implantation.

A bare metal titanium stent is coated with P-15 according to Example 5. Titanium surfaces may also be coated with P-15 through attachment of a ligand or via rapid cooling of the coating solution. The stent can be implanted into a subject to treat a vascular stenosis.

The P-15 coated vascular stents of the invention can reduce local inflammation post implantation, reducing the risk of restenosis in a subject.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. Other embodiments are within the claims.

What is claimed is:

1. A method for fixing two vertebral bodies of a subject, the method comprising:
(a) providing (i) a spinal fusion cage comprising a polyarylether ketone (PAEK) having an internal surface and an external surface, and (ii) inorganic particles coated with P-15 peptide; and
(b) positioning (i) the spinal fusion cage, and (ii) the inorganic particles coated with P-15 peptide between a first vertebral body and a second vertebral body;
wherein step (b) comprising placing the inorganic particles coated with P-15 peptide within the spinal fusion cage and outside of the spinal fusion cage.

2. The method of claim 1, wherein the PAEK is a polyether-ether-ketone (PEEK).

3. The method of claim 1, wherein the PAEK is a polyetherketone (PEK).

4. The method of claim 1, wherein the PAEK is a polyetherketoneketone (PEKK).

5. The method of claim 1, wherein the PAEK is a polyetheretherketoneketone (PEEKK).

6. The method of claim 1, wherein the PAEK is a poly(aryl-ether-ketone-ether-ketoneketone (PEKEKK).

7. The method of any one of claims 1-6, wherein the PAEK has a molecular weight (Mn) of from 110-120 KDa.

8. The method of any one of claims 1-6, wherein the PAEK has a molecular weight (Mn) of from 100-110 KDa.

9. The method of any one of claims 1-6, wherein the PAEK has a molecular weight (Mn) of from 80-100 KDa.

10. The method of any one of claims 1-9, wherein the PAEK has a glass transition temperature of between 300° C. and 380° C.

11. The method of any one of claims 1-10, wherein the PAEK is a composite material comprising fibers and/or a radio-opacity agent.

12. A method for fixing two vertebral bodies of a subject, the method comprising:
(a) providing (i) a spinal fusion cage comprising a polyetherketoneketone (PEKK) having an internal surface and an external surface, and (ii) inorganic particles coated with P-15 peptide; and
(b) positioning (i) the spinal fusion cage, and (ii) the inorganic particles coated with P-15 peptide between a first vertebral body and a second vertebral body,
wherein the PEKK has a glass transition temperature of between 250° C. and 450° C.

13. The method of claim 12, wherein the PEKK has a molecular weight (Mn) of from 110-120 KDa; the PEKK has a molecular weight (Mn) of from 100-110 KDa, or the PEKK has a molecular weight (Mn) of from 80-100 KDa.

14. The method of claim 12 or 13, wherein the PEKK is a composite material comprising fibers and/or a radio-opacity agent.

15. A method for fixing two vertebral bodies of a subject, the method comprising:
(a) providing (i) a spinal fusion cage comprising a polyether-ether-ketone (PEEK) having an internal surface and an external surface, and (ii) inorganic particles coated with P-15 peptide; and
(b) positioning (i) the spinal fusion cage, and (ii) the inorganic particles coated with P-15 peptide between a first vertebral body and a second vertebral body,
wherein the PEEK has a glass transition temperature of between 250° C. and 450° C.

16. The method of claim 15, wherein the PEEK has a molecular weight (Mn) of from 110-120 KDa; the PEEK has a molecular weight (Mn) of from 100-110 KDa, or the PEEK has a molecular weight (Mn) of from 80-100 KDa.

17. The method of claim 15 or 16, wherein the PEEK is a composite material comprising fibers and/or a radio-opacity agent.

18. A method for fixing two vertebral bodies of a subject, the method comprising:
(a) providing (i) a spinal fusion cage comprising a polyetherketone (PEK) having an internal surface and an external surface, and (ii) inorganic particles coated with P-15 peptide; and
(b) positioning (i) the spinal fusion cage, and (ii) the inorganic particles coated with P-15 peptide between a first vertebral body and a second vertebral body,
wherein the PEK has a glass transition temperature of between 250° C. and 450° C.

19. The method of claim 18, wherein the PEK has a molecular weight (Mn) of from 110-120 KDa; the PEK has a molecular weight (Mn) of from 100-110 KDa, or the PEK has a molecular weight (Mn) of from 80-100 KDa.

20. The method of claim 18 or 19, wherein the PEK is a composite material comprising fibers and/or a radio-opacity agent.

21. A method for fixing two vertebral bodies of a subject, the method comprising:
(a) providing (i) a spinal fusion cage comprising a polyetheretherketoneketone (PEEKK) having an internal surface and an external surface, and (ii) inorganic particles coated with P-15 peptide; and
(b) positioning (i) the spinal fusion cage, and (ii) the inorganic particles coated with P-15 peptide between a first vertebral body and a second vertebral body,
wherein the PEEKK has a glass transition temperature of between 250° C. and 450° C.

22. The method of claim 21, wherein the PEEKK has a molecular weight (Mn) of from 110-120 KDa; the PEEKK has a molecular weight (Mn) of from 100-110 KDa, or the PEEKK has a molecular weight (Mn) of from 80-100 KDa.

23. The method of claim 21 or 22, wherein the PEEKK is a composite material comprising fibers and/or a radio-opacity agent.

24. A method for fixing two vertebral bodies of a subject, the method comprising:
(a) providing (i) a spinal fusion cage comprising a polyaryl-ether-ketone-ether-ketoneketone (PEKEKK) having an internal surface and an external surface, and (ii) inorganic particles coated with P-15 peptide; and
(b) positioning (i) the spinal fusion cage, and (ii) the inorganic particles coated with P-15 peptide between a first vertebral body and a second vertebral body,
wherein the PEKEKK has a glass transition temperature of between 250° C. and 450° C.

25. The method of claim 24, wherein the PEKEKK has a molecular weight (Mn) of from 110-120 KDa; the PEKEKK has a molecular weight (Mn) of from 100-110 KDa, or the PEKEKK has a molecular weight (Mn) of from 80-100 KDa.

26. The method of claim 24 or 25, wherein the PEKEKK is a composite material comprising fibers and/or a radio-opacity agent.

27. The method of any one of claims 1-26, wherein the inorganic particles are calcium phosphate particles.

28. The method of claim 27, wherein the calcium phosphate particles are hydroxyapatite particles, anorganic bone mineral (ABM) particles, tricalcium phosphate particles, or admixtures of hydroxyapatite particles.

29. The method of any one of claims 1 to 28, wherein the spinal fusion cage comprises a porous material.

30. The method of any one of claims 1 to 29, wherein the amount of the P-15 peptide bound to the surface of the inorganic particles is from 100 to 1500 ng of P-15 peptide per gram of inorganic particles.

31. The method of any one of claims 1 to 30, wherein the inorganic particles coated with P-15 peptide are suspended in a collagen hydrogel.

32. The method of claim 31, wherein the weight ratio of the inorganic particles coated with P-15 peptide to the collagen is from 50:50 to 95:5.

33. The method of any one of claims 30 to 32, wherein (i) the amount of the P-15 peptide bound to the surface of the inorganic particles is from 200 to 1200 ng of P-15 peptide per gram of inorganic particles, and the weight ratio of the inorganic particles coated with P-15 peptide to the collagen is from 75:25 to 95:5.

34. The method of any one of claims 1 to 33, wherein placement of P-15 peptide in or around the spinal fusion cage reduces local inflammation between two vertebral bodies.

35. The method of any one of claims 1 to 34, wherein placement of P-15 peptide in or around the spinal fusion cage reduces local fibrosis between two vertebral bodies.

36. A method of ameliorating implant-induced inflammation at an implantation site in a subject, the method comprising administering to the implantation site (i) an implantable medical device; and (ii) a substrate coated with P-15 peptide, wherein the substrate is not a calcified substrate.

37. A method of ameliorating implant-induced inflammation at an implantation site in a subject, the method comprising inserting at the implantation site (i) an implantable medical device; and (ii) a substrate coated with P-15 peptide, wherein the implantation site does not comprise bone tissue.

38. A method of ameliorating implant-induced inflammation at an implantation site in a subject, the method comprising inserting at the implantation site (i) an implantable medical device comprising a biodegradable polymer; and (ii) a substrate coated with P-15 peptide.

39. The method of claim 37, wherein the biodegradable polymer is selected from poly(lactic-co-glycolic acid) (PLGA), poly(lactic acid) (PLA), polycaprolactone (PCL), polylactic acid (PLA), polyglycolic acid (PGA), poly(butylene succinate) (PBS), and sucrose acetate isobutyrate (SAIB).

40. A method of ameliorating implant-induced inflammation at an implantation site in a subject, the method comprising inserting at the implantation site (i) an implantable medical device; and (ii) a substrate coated with P-15 peptide, wherein the implantation site is a soft tissue.

41. The method of claim 40, wherein the implantable medical device is implanted into a soft tissue selected from neurological tissue, vascular tissue, oral tissue, ocular tissue, nasal tissue, urogenital tissue, gastrointestinal tissue, biliary tissue, aural tissue, or subcutaneous tissue.

42. A method of ameliorating implant-induced inflammation at an implantation site in a subject, the method comprising inserting at the implantation site an implantable medical device comprising a substrate coated with P-15 peptide, wherein the implantable medical device is a neurologic device, a vascular device, a cardiovascular device, an oral device, an ocular device, a nasal device, a urogenital device, a gastrointestinal device, a biliary device, an aural device, a subcutaneous device, a plastic surgical device, a general surgical device, or a prosthetic device.

43. The method of claim 42, wherein the neurologic device is an electrode, pulse generator, or neurovascular catheter; wherein the vascular device is a vascular stent; wherein the cardiovascular device is a pacemaker, a defibrillator, a coronary stent, a cardiovascular catheter, or a heart valve, optionally wherein the heart valve is a tricuspid valve, a pulmonary valve, a mitral valve, or an aortic valve; wherein the oral device is a tracheostomy tube; wherein the ocular device is an intraocular lens, intrastromal corneal ring segment (ICRS), or ophthalmic catheter; wherein the nasal device is a nasal stent; wherein the urogenital device is a mesh, a contraceptive implant, a hernia mesh, a pelvic mesh, a urinary stent, an artificial urinary sphincter, or a urological catheter, optionally wherein the contraceptive implant is an intrauterine device (IUD) or a birth control implant; wherein the gastrointestinal device is a staple, balloon, sleeve, band, a gastric stimulator, or gastrointestinal catheter, optionally wherein the band is a LINX device; wherein the biliary device is a biliary stent; wherein the aural device is a cochlear implant or ear tube; wherein the subcutaneous device is a drug delivery needle or glucose sensor; wherein the prosthetic device is a prosthetic eye, breast implant, a nose prosthesis, a penile implant, or cosmetic implant; or wherein the breast implant is saline breast implant or a silicone breast implant.

44. The method of any one of claims 36 to 43, wherein the implantable medical device comprises a polyarylether ketone (PAEK).

45. The method of claim 44, wherein the PAEK is polyether-ether-ketone (PEEK), polyetherketone (PEK), polyetherketoneketone (PEKK), polyetheretherketoneketone (PEEKK), or poly(aryl-ether-ketone-ether-ketoneketone (PEKEKK).

46. The method of any one of claims 36 to 45, wherein implantation of the implantable medical device reduces local inflammation at the site.

47. The method of any one of claims 36 to 46, wherein implantation of the implantable medical device reduces local fibrosis at the site.

48. An implantable medical device comprising (i) a biodegradable polymer; and (ii) a substrate coated with P-15 peptide.

49. The implantable medical device of claim 48, wherein the biodegradable polymer is selected from poly(lactic-co-glycolic acid) (PLGA), poly(lactic acid) (PLA), polycaprolactone (PCL), polylactic acid (PLA), polyglycolic acid (PGA), poly(butylene succinate) (PBS), and sucrose acetate isobutyrate (SAIB).

50. An implantable medical device designed for implantation into soft tissue and comprising a substrate coated with P-15 peptide.

51. The implantable medical device of claim 50, wherein the implantable medical device is a neurologic device, a vascular device, a cardiovascular device, an oral device, an ocular device, a nasal device, a urogenital device, a gastrointestinal device, a biliary device, an aural device, a subcutaneous device, or a prosthetic device.

52. The implantable medical device of claim 51, wherein the neurologic device is an electrode, pulse generator, or neurovascular catheter; wherein the vascular device is a vascular stent; wherein the cardiovascular device is a pacemaker, a defibrillator, a coronary stent, a cardiovascular catheter, or a heart valve, optionally wherein the heart valve is a tricuspid valve, a pulmonary valve, a mitral valve, or an aortic valve; wherein the oral device is a tracheostomy tube; wherein the ocular device is an intraocular lens, intrastromal corneal ring segment (ICRS), or ophthalmic catheter; wherein the nasal device is a nasal stent; wherein the urogenital device is a mesh, a contraceptive implant, a hernia mesh, a pelvic mesh, a urinary stent, an artificial urinary sphincter, or a urological catheter, optionally wherein the contraceptive implant is an intrauterine device (IUD) or a birth control implant; wherein the gastrointestinal device is a staple, balloon, sleeve, band, a gastric stimulator, or gastrointestinal catheter, optionally wherein the band is a LINX device; wherein the biliary device is a biliary stent; wherein the aural device is a cochlear implant or ear tube; wherein the subcutaneous device is a drug delivery device, or glucose sensor; wherein the prosthetic device is a prosthetic eye, breast implant, a nose prosthesis, a penile implant, or cosmetic implant; or wherein the breast implant is saline breast implant or a silicone breast implant.

53. The implantable medical device of any one of claims 48 to 51, wherein the implantable medical device comprises a polyarylether ketone (PAEK).

54. The implantable medical device of claim 52, wherein the PAEK is polyether-ether-ketone (PEEK), polyetherketone (PEK), polyetherketoneketone (PEKK), polyetheretherketoneketone (PEEKK), or poly(aryl-ether-ketone-ether-ketoneketone (PEKEKK).

55. A vascular stent having a surface comprising a substrate coated with P-15 peptide.

56. The vascular stent of claim 55, wherein the stent comprises stainless steel, cobalt-chromium alloys, nickel-titanium alloy, platinum, or tantalum alloys coated with P-15 peptide.

57. A spinal fusion cage comprising a substrate coated with P-15 peptide, wherein the substrate is not a calcified substrate.

58. The spinal fusion cage of claim 57, wherein the spinal fusion cage comprises a polyarylether ketone (PAEK).

59. The spinal fusion cage of claim 58, wherein the PAEK is polyether-ether-ketone (PEEK), polyetherketoneketone (PEKK), or poly(aryl-ether-ketone-ether-ketoneketone (PEKEKK).

60. The spinal fusion cage of any one of claims 57 to 59, wherein the spinal fusion cage comprises a polyarylether ketone (PAEK) coated with P-15 peptide.


SEQUENCE LISTING (1 sequence)

SEQ ID NO: 1 - protein, 15 aa, synthetic construct
GTPGPQGIAGQRGVV